(19) United States
(12) Patent Application Publication
(10) Pub. No.: US 2007/0059690 A1
(43) Pub. Date: Mar. 15, 2007

(54) "Met/fret based method of target nucleic acid detection whereby the donor/acceptor moieties are on complementary strands"

(76) Inventors: Amirul Islam, Hyderabad (IN); Papia Hazea, Hyderabed (IN)

Correspondence Address: STEPHAN A. PENDORF, P.A., PENDORF & CUTLIFF, 5111 MEMORIAL HIGHWAY, TAMPA, FL, 33634, US

(21) Appl. No.: 10/516,361

(22) PCT Filed: May 30, 2003
(86) PCT No.: PCT/IN03/00204

§ 371 Date: Nov. 30, 2004

(30) Foreign Application Priority Data
May 31, 2002 (IN) 487/MUM/2002

(51) Int. Cl. C12Q 1/68; C12P 19/34

(52) U.S. Cl. 435/6; 435/91.2

(57) ABSTRACT

Disclosure of a method for the detection and quantitation of polynucleotide sequences in a sample of biological or non-biological material through target poly nucleotide sequence amplification by polymerase chain reaction using chemically labeled oligonucleotide amplification primers and formation of an entity between the amplified polynucleotide sequence and chemically labeled polynucleotide having a sequence complementary to the target polynucleotide sequence for determining the identity and/or presence and/or quantitation of the target poly nucleotide sequences. The chemical label covalently attached to the oligonucleotide amplification primer and polynucleotide or oligonucleotide comprise molecular energy transfer labels (donor and acceptor). It is again a very sensitive, rapid and reliable method with better sensitivity, specificity and reliability for the detection of polynucleotide sequence. It also greatly reduces the possibility of amplification product carry-over contamination and adaptable for many formats of nucleic acids amplifications and real time measurements.

### FIELD OF INVENTION

The present invention relates to a method of detection of a target nucleic acid sequence by nucleic acid amplification reaction and to a kit used for such detection of target nucleic acid sequence. It would be possible by way of the above method to detect and quantify polynucleotide sequences in a sample of biological and/or non-biological material by way of a very sensitive, rapid and reliable method with improved specificity and reliability for the detection of polynucleotide sequence.

### BACK GROUND OF THE INVENTION

The analysis and detection of minute quantities of substances in biological and non-biological samples has become a routine practice in chemical diagnostic and analytical laboratories. These detection techniques can be divided into two major classes. (1) Those based on ligand-receptor interaction (e.g. immunoassay-based techniques), and (2) Those based on nucleic acid hybridization (polynucleotides or oligonucleotide sequence-based techniques).

Immunoassay-based techniques involve a sequence of steps based on non-covalent binding of an antibody and antigen complementary to it. In these techniques analytes of concentration as low as a nanomole can be detected.

The present trend is towards the detection of polynucleotide sequences for analyte analysis. Polynucleotide sequence based detection of analytes requires detection limit as low as attomole. Polynucleotide sequence based techniques are mostly based on hybridization, the non-covalent binding in accordance with Watson-Crick base pairing of a labeled polynucleotide sequence to a complementary sequence of the analyte. Such polynucleotide sequence based detection techniques are divided into two categories: (1) Heterogeneous phase detection, (in which the analyte is fixed to a solid phase support such as nylon, cellulose etc., the labeled oligonucleotide is hybridized to the analyte, are washed in a number of steps and finally detected by colorimetric/color precipitation/Chemiluminescence/bioluminescence/fluorescence/ELISA), and (2) Homogeneous phase detection, in which detection is carried out in solution.

Heterogeneous phase detection techniques normally give higher sensitivity, i.e., detection of lower quantity of the analyte in comparison to homogeneous phase detection. But heterogeneous phase reaction is slow and more over involve many washings and other separation steps before final detection; hence those are more time consuming and complex. On the other hand, homogeneous phase detections are very simple, fast, easy to automate, easy to handle and adapt in any laboratory. Only disadvantage is its lower sensitivity. The detections are mostly fluorescence spectrophotometry based. With the advent of polymerase chain reaction, RT-PCR, NASBA and ligase chain reactions, which are again homogeneous phase techniques, the target polynucleotide/oligonucleotide sequence can be amplified 10⁶-10⁸ times, thus even a less sensitive detection method coupled with a target polynucleotide sequence amplification method can give very high sensitivity. Hence, a homogenous phase detection method in conjunction with any of the above nucleic acid amplification techniques is ideal for detection and quantification of polynucleotide/oligonucleotide sequences in analyte. Molecular energy transfer and particularly, fluorescence resonance energy transfer (FRET), based detection methods are ideal for homogenous phase detection.

FRET labels were first introduced in 1970's in immunofluorescence assay for detection of specific antigen (Ulman et al J. Biochem (1970), 251, 4172-4178, U.S. Pat. Nos. 2,998,943; 3996,345; 4160,016; 4174,384; and 4,199,559). Later in the 1980's many methods of detecting DNA and RNA by homogenous sequence specific hybridization using energy transfer and fluorescence quenching labels were developed (Heller et al U.S. Pat. Nos. 4,996,143; 5,532,129; and 5,565,322; European patent No. 070,685; year 1983 and others). In European patent 070, 685, year 1983 “Light emitting polynucleotide hybridization diagnostic method”, Heller et. al. described the detection of a longer single stranded DNA target using two oligonucleotide probes, one labeled with a donor fluorophore at 5′ end and the other labeled with an acceptor at its 3′ end such that on hybridization two labels are placed close resulting in fluorescence energy transfer.

In European patent 229, 943, year 1987, “Fluorescent strokes shift probes for polynucleotide hybridization assay”, Heller et al described the same scheme with specified distances between the donor and acceptor for maximum FRET. They also disclosed that the donor and acceptor could be located on the same probe.

A later demonstration used oligonucleotides with terminal labels (5′ and 3′) of fluorescein and tetramethyl, rhodamine (Cardullo et.al. 1988, Detection of nucleic acid hybridization by non-radioactive fluorescence resonance energy transfer, Proc. Natl. Acad. Sci. USA., 85, 8790-8794). Fluorescence of fluorescein label was reduced 71% on addition of the target DNA. In a recent variation on this format, a strong metal chelator (fluorescent acceptor) was attached to the 5′-terminus of one oligonucleotide and a weaker fluorescent chelator (donor) was attached to the 3′-terminus of the second oligonucleotide (Oser and Valet, 1990, Non-radioactive assay of DNA hybridization by DNA template mediated formation of a ternary Tb (III) complex in pure liquid phase, Angew Chem. Int. Ed.Engl. 29,1167-69).

WO 92/14845 entitled, “Diagnosing cystic fibrosis and other genetic diseases using fluorescence resonance energy transfer” discloses a DNA hybridization based detection system similar to that of Heller et. Al (European patent 070,685; year 1983).

In a second assay format referred to as a competitive assay one probe is labeled on its 3′ terminus, and the other probe is labeled on its 5′ terminus and they hybridize to each other resulting in fluorescence quenching (European patent 232,967; year 1987, Morrison et. al. Solution phase detection of polynucleotides using interacting fluorescent labels and competitive hybridization, Anal. Biochem. 183, 231-244). In target detection there is competition between the probes and the target. More the target strands present, more the probe strands hybridize to the target strands and lesser the number of donor and acceptor placed next to one another by probe to probe hybridization. The presence of target DNA is detected as increased emission from donor due to reduced quenching, and reduced emission from acceptor due to reduced energy transfer.

In “Rapid detection and identification of infections agents” pages 245-256 in Chemiluminescent and fluorescent probes for DNA hybridization systems ed. by D. T. Kingsbury and S. Falkow, Academic press; Heller and Morrison described another nucleic acid detection format in which one fluorophore (F) labeled single stranded probe and a dye (Q) that preferentially binds to double stranded DNA is used. In presence of the target DNA, the probe hybridizes to the target and acceptor (Q) binds to the resulting double helical region, thereby placing Q near F and allowing fluorescence energy transfer or fluorescence quenching.

FRET has also been used for studying the hybridization process (Morrison and Stols 1989, The application of fluorophore labeled DNA to the study of hybridization kinetics and thermodynamics, Biophys. Jl, 55, 419; A sensitive fluorescence based thermodynamic and kinetic measurement of DNA hybridization in solution, Biochem. 32, 3095-3104, Perkins et. al., 1993, Accelerated displacement of duplex DNA strands by a synthetic circular oligodeoxynucleotide, J Che. Soc. Chem. Comm. 215-216).

Distance relationship in double helical structures has also been measured using energy transfer between two fluorescent labels attached to DNA oligomers (Cardullo et.al. 1988, Proc. Natl. Acad. Sci. USA, 85, 8790-8794; Cooper and Haserman, 1990, Analysis of fluorescence energy transfer in duplex and branched DNA molecules, Biochem. 29, 9261-9268, Ozaki and Mc. Laughlin, 1992, The estimation of distance between specific backbone-labeled sites in DNA using fluorescence resonance energy transfer, Nucl. Acids Res, 20, 5205-5214; Clegg et. al. Observing the helical geometry of double stranded DNA in solution by fluorescence resonance energy transfer Proc. Natl Acad. Sci. U.S.A, 90, 2994-2998).

For clinical and other analyses, detection requirement below, one attomole of polynucleotides are common. Fortunately, there are polynucleotide amplification systems available that amplify the target polynucleotides. Among those are polymerase chain reaction (PCR), reverse transcription coupled polymerase chain reaction (RT PCR), ligase chain reaction (LCR), Nucleic acid sequence based amplification (NASBA), Triamplification, Strand displacement amplification (SDA) and others. The PCR•(Mullis and Faloona, 1987, Methods in Enzymology 155, 335-350) is the best known and most studied of the amplification systems. All the above nucleic acid amplification methods are capable of producing well over a million copies of the target polynucleotide originally present in the sample.

Commonly nucleic acid amplification product detection requires the separation of the product from the unreacted primers and nucleotides. Agarose gel electrophoresis is the most commonly used technique for this and is based on size differentiation. The detection is by ethidium bromide staining of the gel. Alternatively, the amplification product is immobilized on a solid surface and detected with a labeled product. The unreacted primers, probes and nucleotides are washed always. One of the problems associated with the detection of the amplified product by the above two methods is carry over contamination of the amplified product. Since there is high level of target sequence amplification, while opening the tubes containing the amplified product, the amplified product gets released in the aerosol form and contaminates the laboratory. Subsequent amplification reactions will have the contamination of this target sequence (because of high amplification), thus giving false positive results. However, MET/FRET allows detection of amplification product without separation of the unutilized primers, probes and nucleotides. Hence there is no need to open the amplification reaction tube and no carryover contamination problem. Moreover, MET/FRET is solution phase homogeneous detection technique, hence very simple, fast and efficient detection method, and amenable to automation.

The first method reported for the detection of amplification product without prior separation is based on the 5′-exonuclease degradation of doubly labeled probe during PCR amplification, referred to as the Taq Man assay (Holland et. al., 1991, Proc. Natl Acad Sci U.S.A. 88, 7276-7280; Lee et. al., 1993, Nucl. Acids. Res, 21, 3761-3766. In this assay during the annealing step of the amplification reaction the doubly labeled fluorogenic probe hybridizes to the complementary target sequence. The 5′-exonuclease activity of the enzyme Taq DNA polymerase used for amplification degrades the hybridized probe. The probe is degraded only when it hybridizes to the target sequence being amplified. One of the labels is a fluorescent donor and the other is a quencher. In the labeled probe fluorescence of the donor is quenched. On degradation of the probe quenching is removed and the donor fluorophore fluoresce resulting in the detection of the target. In Taq man assay the donor and the quencher are located preferably at the two ends of the probe, i.e., the 5′ and 3′ ends; because the 5′ to 3′ exonuclease hydrolysis of the probe can be achieved only when these two labels are not too close to each other (Lyamichev et.al 1993, Science, 260, 778-783). This is a serious drawback of this assay method. The efficiency of energy transfer between the donor and the quencher (acceptor) decreases with increase of distance between two by inverse sixth power of the distance. Since, the quencher cannot be placed close to the donor, most efficient quenching of the donor (reporter) cannot be achieved. As a result, background fluorescence from unhybridized probes will be high.

Further in Taq man assay the amplification product is not measured directly rather an event related to the amplification, i.e., the hydrolysis of the probe that hybridizes to the amplification product between the two primer sequences. Following problems are associated with this method as discussed in the U.S. Pat. No. 5,866,336. First, hybridization will never be quantitative unless the labeled oligonucleotide probe is in great excess. This in turn will result in high background (because quenching is never quantitative and moreover Taq man probes are not quenched efficiently). Secondly, oligonucleotide probes hybridized to the middle of the target DNA will slow down the PCR amplification process. Thirdly, all of the oligonucleotide probes hybridized to the amplified product will not be subjected to 5′-3′ exonuclease hydrolysis; some will be displaced without hydrolysis resulting in loss of signal. Fourthly, probes non-specifically hybridized to the portion of the amplified product other than the targeted region will give fluorescence signal resulting in over estimate of the analyte.

Another method of detection of amplification product using FRET is the molecular beacon probe method described by Tyagi and Kramer, 1996, Nature Biotech. 14, 303-309, Lizardi et.al. U.S. Pat. No. 5,312,728). This method is again based on oligonucleotide probe hybridization. The oligonucleotide probes (molecular beacons) are of hair-pin (loop and stem) configuration. On one end of the oligonucleotide probe (either 5′ or 3-end) there is a donor fluorophore, and on the other end an acceptor moiety, which is a quencher.

The molecular beacon probes are in strained conformation. Whenever, the loop portion contact perfectly matched target sequence it forms a stable hybrid destabilizing the stem structure, resulting in an open conformation of the probe separating the donor fluorophore from the acceptor (quencher). Otherwise, in the absence of the target sequence, the beacon probe is in its closed conformation (hair-pin), in which the fluorescence of the donor fluorophore remains quenched.

When employed in PCR assay, the molecular beacons, which hybridize to one strand of PCR products are in open conformation and emit detectable fluorescence. Those molecular beacons that remain unhybridized will not fluoresce. The amount of fluorescence will increase as more and more PCR products are formed, giving a measure of the progress of PCR and ultimately measure of the analyte in the sample.

Since, this method is solely based on probe hybridization like Taq man assay it also has the drawbacks of hybridization methods. Though high specificity and sensitivity is claimed, there still remains certain amount of non-specificity. It is unlikely that the beacon probe will quantitatively hybridize to the particular strand and particular site only for which it is designed, particularly when the PCR product is much longer than the beacon. It can also hybridize to other non-template nucleic acid sequences present in the sample and to non-specifically amplified products.

Even those probes that are hybridized to the template could be displaced by the second DNA strand under synthesis or polymerization during annealing or annealing cum extension step (two step amplification i.e., denaturation and extension required for some amplifications) over a short period of time; as a result this method cannot be quantitative.

Another major draw back of the method is that the measurement is based on removal of quenching of the donor fluorophore. Quenching can never be quantitative. As a result the fluorescence background will be high. Since the method is based on hybridization of the probe to the amplified product, quantitative hybridization of the probe will require higher concentration of probe that in turn will increase fluorescence background further (as discussed in U.S. Pat. No. 5,866,336; year 1999). In addition dissociation of the donor or the quencher from the probe during PCR process due to the break down of the linkage between the probe and the fluorophore and/or the quencher will increase the nonspecific signal background resulting in low signal to background ratio and thus limiting detection limit (lower sensitivity of detection). Further beacon probes are susceptible to degradation by exonuclease activities of the polymerase thus resulting in separation of the flurophore and the quencher and increasing the background noise. In this method primers are designed to amplify a product closed to 100 base pair for hybridization of the beacon probe. Because of size constraint designing good primers will be difficult which will result in non-specific product formation. This method does not specify about the position where the probe hybridizes. If the probe hybridizes close to the primer being extended or in the middle there will be inhibition of PCR reaction, which will result in lower yield, hence lowering of signal, higher non-specific product formation resulting in higher background noise.

Non-specific products do form in PCR nucleic acid amplification reaction at some stage or other particularly with complex samples. Some non-specific products can get extended from its 3′ end over the 3′ end of the labeled hair-pin beacon probe up to the 5′ end of the same. This extension through the hair-pin probe would be in right orientation and can anneal with the target sequence and extend in the next cycle of the PCR amplification, to the respective end of the amplification product, the resulting product can be amplified exponentially thereafter. Thus a non-specific amplification product generated at some stage of amplification can result in exponentially amplified product to which hair-pin beacon probes can hybridize thus giving a higher estimate of the target sequence.

Moreover, for multiplexing, i.e., detection of multiple targets in single reaction tube will require multiple light sources and hence costlier instrument.

In 1995, Jingyere Ju et. al. PNAS 1995, 92, 4347-4351, (U.S. Pat. No. 5,707,804, 1998) developed fluorescence energy transfer primers for DNA sequencing. In 1995, Wang et. al. Anal chem. 1995, 67, 1197-1203) used the fluorescence energy transfer primers in PCR detection of simple tandem repeat sequences. In both the methods fluorescent energy transfer (FRET) primers were incorporated into the PCR amplification product. However, their objective was to improve the fluorescence intensity of non-radioactive sequencing ladder and to detect different repeat lengths in individual samples respectively. Resolution of different repeat lengths requires separation and they used capillary electrophoresis for the same.

Wang et al in U.S. Pat. No. 5,348,853 described a fluorescence energy transfer based method for the detection and quantitation of nucleic acid target using PCR amplification. In this method an asymmetric PCR amplification of a target sequence was carried out using one amplification primer in excess such that one of the target strands is significantly overproduced. A primer duplex labeled with a donor and an acceptor fluorophores, complementary to the over produced target was used to prime a semi-nested reaction in concert with the excess primer. As the semi nested amplification proceed the labeled reverse primer start dissociating from the primer duplex and getting incorporated into the amplified product. The incorporation of the labeled reverse primer into the amplification product was measured by the disruption of the energy transfer between the donor and the acceptor of the primer duplexes and through the decrease in the fluorescence intensity of the acceptor. The decrease in the fluorescence intensity was proportional to the initial target dosage and the extent of amplification. The method depended on decrease in emission from the acceptor fluorophore rather than the increase in donor fluorophore emission. Hence the signal to noise ratio was low. Moreover the method used a preliminary amplification step (asymmetric PCR) to increase the initial target concentration and subsequent addition of labeled primer duplex which complicates the process as well as involves opening of the tube.

Another method Nazarenko et.al. U.S. Pat. No. 5,866,336 and US 6,117,635 which is a modification of the Wang et al. (U.S. Pat. No. 5,348,853) disclosure of a method based on the incorporation of a fluorophore labeled reverse primer into amplification product and indirect measurement of the same through decrease in acceptor emission rather than direct measurement through increase in donor emission based on FRET. The method is based on the incorporation of FRET primer/primers into the PCR amplification product. One of the two amplification primers is a FRET primer, i.e. labeled with a fluorescent donor moiety and an acceptor moiety; the acceptor moiety can be another fluorophore or quencher. The detection and quantitation is based on the incorporation of the fluorescently labeled primer into the PCR amplification product and thereafter excitation of the donor fluorophore. The FRET primer either does not fluoresce or fluoresce at wavelengths different from that of the donor when it is not incorporated into an amplification product. Here PCR amplification product is measured directly by measuring the amount of fluorescence emitted by those products into which the fluorescently labeled primer has been incorporated.

The above method also has many drawbacks. Firstly the preferred detection primer is a hair-pin quenched primer and fluorescence signals are generated by removal of quenching as a result of incorporation of the primer into the amplification product. Due to non-quantitative nature of quenching there will be higher background.

Secondly, the preferred hair-pin primers used have long non-target specific sequence (from universal hair-pin configuration) at the 5′ end of the target specific primer (Primer-1). Addition of non-target specific sequence at 5′ end of a specific primer brings non-specificity due to the fact that at the annealing temperature of the target specific primer, the primer with added sequence may anneal in many other places on the complex genomic materials of the sample (which may contain a mixture of one or more contaminating genomic material in addition to the genomic material of the target sequence) resulting in non-specific product; while increase in annealing temperature to avoid non-specific product formation may result in failure of the amplification reaction. Further hair-pin primers are susceptible to nuclease degradation which will result in separation of the donor fluorophore from the acceptor fluorophore or quencher resulting in higher background noise. This may be a major reason for low signal to noise ratio (35)achieved. In addition this method requires higher magnesium ion concentration (2.5 mM) for stabilizing the hair-pin stem structure. This higher concentration of magnesium again will result in non-specific amplification product formation and lower signal to noise ratio. Thus there will be an over all decrease in signal to noise ratio and hence decrease in sensitivity of target detection.

Primer dimer formation by the hair-pin labeled primer of the preferred embodiment with itself (homodimer) or the second primer (heterodimer) will generate signal resulting in background. Primer dimer formation is a concern for any nucleic acid target detection based on incorporation of labeled primer (provided quenched in unincorporated condition) into an amplification product. Moreover, any non-specific product formed during any stage particularly at the beginning of amplification can get extended through the labeled hair-pin primer, the way primer dimers are formed, resulting in product that amplifies exponentially in the remaining cycles thus resulting in high background and low signal to noise ratio.

Designing a good suitable amplification primer with long non-target specific sequence at the 5′-end of target specific primer sequence not giving non-specific product for amplifying a target from a complex sample, such as genomic DNA, total RNA from bacteria, fungi, plants and animals is difficult or limited. And taking into account the possibility of contamination from many of these materials in the sample, job is much more difficult. This is a limitation of the method. There may be a success in case of a specific target in a specific sample but in general there will be problem in the amplification. Moreover, in view of the practical problem of temperature variation from well to well in the same thermal cycler, between thermal cycler to thermal cycler and laboratory to laboratory, amplifying a specific target polynucleotide sequence with the above type of primers free of any non-specific amplification product would be difficult and limited. Further higher quantity of nucleic acid as well as degraded nucleic acid in a sample result in formation of non-specific amplification products. As in this method the quantitation is based on incorporation of the above-labeled primer into the amplification product, hence there is bound to be an over estimate of the target due to formation of non-specific amplification product. Also any primer extension specific or non-specific resulting either in amplification product or abortive amplification will result in signal generation thus giving an over estimate of the target. Avoiding non-specific amplification product in PCR amplification is real problem. On the other hand use of higher annealing temperature to increase specificity of amplification to overcome above problem will result in amplification failure and hence lowering of sensitivity of detection of the target.

The amplification in PCR is tremendous 10⁶times in 20 cycles. That is why PCR amplification based detection is a highly sensitive method. Because of this high level of amplification specificity of amplification needs to be highly specific. High stringency annealing of the primer increases specificity. Again, higher the specificity, higher is the PCR amplification failure, hence lesser overall sensitivity of the method. Addition of the thermostable DNA polymerase enzyme in the PCR little more than the required give a lot of non-specific product and primer dimer; and it is difficult to add always exactly the required quantity of the enzyme since the enzyme comes in 50% glycerol solution and mostly in higher concentration (5,000 units/ml). Dispensing of 50% glycerol solution always result in higher volume of the dispensed solution. Here, is also the scope for personal error. It is also observed that different preparations of thermostable DNA polymerases from different sources give different amounts of non-specific amplification products. The present method is solely, dependent on the incorporation of labeled primer into the amplification product; taking this and non-specificity of amplification particularly with the said hair-pin primer, into account the method will give an over estimate of the target.

Background problem associated with the use of higher concentration of labeled molecular beacon probes (Tyagi and Kramer, 1996, Nature Biotech, 14, 303-309; Lizardi et al U.S. Pat. No. 5,312,728), which are normally used at a concentration of 0.5 □M is also known. The same background problem will be there with the use of labeled amplification primers, which are normally used at a concentration of 0.2-0.5 □M (range 0.1-1 □M). Only 1 to 20 percent of the primers normally get incorporated into the PCR amplification product. Such a large excess of primers will give high background because fluorescence quenching in the primers cannot be quantitative. During the PCR amplification a certain percentage of the fluorophore/quencher or donor/acceptor whatever may be the case will get detached from the labeled amplification primer due to the breakdown of the linkage between the fluorophore and/or the quencher or the donor/acceptor as the case may be, thus resulting in background, inaccuracy in quantitation and rise in detection limit i.e., lower sensitivity. Moreover in preferred embodiment multiplexing, i.e., detection of multiple targets in single reaction tube will require multiple light source and hence costlier instrument.

In case of triamplification a blocking primer and one of the amplification primers complementary to the blocking primer are labeled separately with a donor and an acceptor in order to give FRET when not incorporated into the amplification product. The acceptor is a fluorophore and reduction in sensitized emission from the acceptor is measured for detection and/or quantitation. Problem in using an acceptor fluorophore in FRET is that the acceptor fluorophore gets excited to sizeable extent by the light used for exciting the donor thus resulting in considerable background. This is a major problem in measurement based on sensitized emission of an acceptor moiety. Hence both in case of 5′→3′ exonuclease degradation and heating at high temperature used for detection of the amplification product there will be sizeable background because of this excitation of the acceptor by the donor excitation light. The same problem will also be there incase where linear amplification primers doubly labeled with a donor fluorophore and an acceptor fluorophore is used, even after 5′→3′ exonuclease degradation of the unutilized primers, (which is in much excess) the acceptor fluorophore released from the primer will be partially excited by the light used for excitation of the donor fluorophore, thus resulting in sizeable background (Morrison, L. E. In the chapter “Detection of energy transfer and fluorescence quenching” in the book, Nonisotopic probing, blotting and sequencing, 1995, pages 442, 444, 445, 490 edited by Kricka L J and published by Academic Press.

FRET in triamplification, and use of linear FRET primers in amplification in conjunction with 5′→3′ exonuclease digestion and heating at higher temperature for the detection and quantitation of polynucleotide target is proposed but use of FRET between donor and acceptor moieties on two amplification primers have not been put forward.

In U.S. Pat. No. 6,037,130 Tyagi et al. discloses the use of wavelength shifting hair-pin primer and probe. The above said primer and probe are triple labeled with a harvester, an acceptor and a quencher. The harvester absorbes light and emits energy at a wave length or wavelengths which is transfered to the acceptor which remains quenched by a quencher in the closed configuration of hair-pin oligonucleotide primer or probe and emits light when incorporated into or hybridized to the amplification product. The above said wave length shifting hair-pin primer and probe are an extension of the hair-pin quenched amplification primer (Nazarenko et.al. U.S. Pat. No. 5,866,336 published in year 1999 and U.S. Pat. No. 6,117,635 in year 2000) and hair-pin quenched probe (Tyagi and Kramer, 1996, Nature Biotech. 14, 303-309, Lizardi et.al. U.S. Pat. No. 5,312,728) as discussed above.

U.S. Pat. Nos. 6,140,054 & 6,174,670 involve use of FRET for the detection or quantitation of nucleic acid target sequence and mutation detection in particular. In this method two hybridization probes one labeled at its 3′ end and the other labeled at its 5′ end separately with a donor or an acceptor FRET moiety is used so that when these two probes hybridize to the strand of the amplification product against which these two probes are configured the donor and the acceptor moieties on two probes come in proximity such that FRET can take place between the two moieties and measurement of the increase in acceptor emission or decrease in donor emission gives the measurement of the amplification product or the progress of the amplification process. Also mentioned is the use of one of the amplification primers as one of the above two hybridization probes. This method also has a lot of drawbacks. In this method two probes configured for hybridization to one strand of the amplification product will hamper the amplification reaction resulting in sluggish PCR amplification reaction and non-specific product formation. The probe/probes can easily be displaced by the polymerase before the FRET signal can be measured thus resulting in a lower estimate of the amplification product or the amplification reaction. In this method there will be a lot of background because of the excitation of the acceptor FRET moiety by the light used for exciting the donor fluorophore and higher concentration of the probes. Because of this background the method can be used for a very limited number of donor acceptor pair and sensitivity of target nucleic acid detection will be low.

In Nature Biotechnology (2001) 19,365-370 use of nano gold particle as quencher for using in oligonucleotide probe has been reported. It has been claimed that the nano gold particles can quench donor fluorophore emission to the extent of 99.9% i.e. the background fluorescence will be very low almost 20 fold less in comparison to the prior art methods. However, a small percentage of non-specific product formation or breakage of the linkages linking the quencher and the fluorophore or degradation of the probe by exonucleose activity of the polymerase will bring down the signal to noise ratio to a large extent to the level of 25-50.

In U.S. Pat. No. 6,323,337 Singer et al disclosed a method for nucleic acid target detection using luminescent nucleic acid stains like acridine, indole, cyanine dye etc. and an oligonucleotide labeled with an acceptor (quencher) is provided for reducing the background of the nucleic acid stain. Nucleic acid stain based target detection methods will not be as sensitive as a FRET based method. Further acceptor labeled oligonucleotide is susceptible to exonuclease activities of polymerase, which will increase the background luminescence.

Gildea Brian D etal in U.S. Pat. No. 6485,901 have disclosed a probe based method for target nucleic acid detection using linear beacon probe, which is a PNA (protein nucleic acid) molecule labeled with a donor and an acceptor MET moiety at two ends and which in its solvated state in aqueous phase result in FRET between the donor and acceptor moieties independent of probe length, spectral overlap between the donor and the acceptor, magnesium ion concentration and ionic strength of the solution. On hybridization of the above probe to the target sequence there is a measurable change in at last one property of at lease one of the donor or acceptor moiety of the probe which can be used for detection or quantitation of target sequence in a sample. PNAS are known to exhibit resistance to exonuclease degradation thus the above linear beacon probes will result in less background. This method is also based on quenching of a donor fluorophore and quenching can never be quantitative. The method can be used for real time quantitation of target nucleic acid sequence but nothing is mentioned about the signal to noise ratio and detection sensitivity is as good as that of molecular beacon probe method (Tyagi et.al. 1996 Nature Biotech 14,303-309).

In U.S. Pat. No. 6,485,903, Mayrand has disclosed a method of use of hybridization probe in PCR amplification for target detection by providing a duplex labeled probe impervious to digestion by 5′ to 3′ and 3′ to 5′ exonuclease activities of polymerases and using a strand displacer for facilitating amplification process, i.e, removing inhibition of PCR reaction associated to probe hybridization. This method addresses two of many problems associated with hybridization based method one is degradation of probe by exonuclease activity of polymerase which is otherwise resistant to exonuclease digestion by modifying the two ends of the probe by incorporating modified internucleotide linkage and strand displacer for facilitating amplification process. Polymerases like vent/deep vent polymerase, Pfu polymerase etc. have strand displacement activity. Use of strand displacer in association to a probe designed to hybridize close to the primer which is being extended on the strand on which the probe sits will result in loss of signal due to displacement of the probe before the measurement of the signal. Problem associated to non-specificity and higher background from higher probe concentration and proper quenching required still remain.

In U.S. Pat. No. 6,495,326 Dr. Kurane etal uses oligo probe labeled with a fluorophore. The probe is so designed that when said probe is hybridized to target nucleic acid at least one guanine base exists in base sequence of said target nucleic acid at position 1 to 3 from the end base position where the said probe and target hybridize. The probe is labeled with a fluorophore at its one end. On hybridization of the probe to the target sequence result in quenching of the fluorescence of the fluorophore to the extent of maximum 90%. This is again a probe based method and quenching of the fluorophore is measured as signal. This method has the defects associated to a probe based target detection method and has a low signal to noise ratio.

In U.S. Pat. No. 6,534,274 Becker et al has disclosed a method for detection of target nucleic acid sequence based on hybridization of a molecular torch that contains a target binding domain, a target closing domain and a joining region. The target-binding domain is biased towards the target sequence such that the target-binding domain forms a more stable hybrid with the target sequence than with the target-closing domain under the same hybridization condition. Molecular torches are suitably labeled with a FRET pair. The joining region facilitates the formation or maintenance of a closed torch in absence of target sequence and generation of FRET signal in presence of target sequence in strand displacement condition. This is again a hybridization probe based method and has all the problems associated to hybridization probe based nucleic acid detection methods discussed earlier.

In U.S. Pat. No. 6,534,645 Mc Millan, W. A., has disclosed a method for performing a amplification reaction with internal control by providing an internal control template, primer pair and a probe for the same, and sample template, primer pair and a probe for the sample template, the probes being labeled with a fluorophore and a quencher. This method teaches about use of control template for monitoring of the performance of the specific PCR reaction and quantitation of the target template in the sample by means of comparison but the method has all the defects associated to hybridization probe based nucleic acid detection methods discussed earlier.

It is clear from the prior art methods for detecting amplification products that PCR based detection methods are simple, rapid and highly sensitive. However, because of high level of amplification of nucleic acid in such PCR based detection there is associated carryover contamination problem. One way of avoiding this is not to open the reaction tube for detection, i.e. to adapt a close tube detection format, which can be achieved by using FRET, based detection. All the FRET based methods partly use the FRET probe or primer where both the donor and the acceptor moieties are part of the same oligonucleotide. In case of hair-pin quenched probe or primer the donor and the acceptor moieties are on the same oligonucleotide but the two MET moieties are placed opposite to each other in the two opposite strands of the stem of the hair-pin structure of the probe or primer. In such known FRET based detection methods the measurement is based on removal of fluorescence quenching and lead to non-specific signals due to non-specific incorporation of MET primer into the amplification product or non-specific hybridization of MET probe to the amplification product and detachment of labels from primer or probe. Moreover, the known FRET based methods also suffer from problems of fluorescence/emission background which give relatively high background and signal to noise ratio between twenty five to forty.

Also, any detection or quantitation method may have success in certain cases but for it to be adapted universally in many laboratories need to have proper control. None of the above known methods whether it is molecular beacon method or labeled primer incorporation method meet this requirement. Hence there is need for the development of a FRET based close-tube format involving simple direct low back ground, highly specific, highly sensitive quantitative and reliable method independent of personal error and sample type for the detection of the PCR amplification product.

### OBJECTS OF INVENTION

It is thus the basic object of the present invention to provide for an improved sample of biological and/or non-biological material through target polynucleotide sequence method for the detection and/or quantitation of polynucleotide sequences in amplification involving polymerase chain reaction which would be very sensitive, rapid and reliable method and bring about better sensitivity, specificity and reliability in the detection of polynucleotide sequences.

Another object of the present invention is to provide for a method of detection and/or quantitation of polynucleotide sequences which would substantially avoid the problems of known FRET based detection techniques and thereby provide for an effective PCR based detection method.

Another object of the present invention is to provide for the detection and/or quantitation of polynucleotide sequences in sample of biological and/or non-biological material through target polynucleotide sequence amplification involving FRET in a closed tube format which will reduce the possibility of carry over contamination whereby the measurement can be carried out in real time both in homogeneous solution phase assay and semi-homogeneous/heterogeneous phase assay.

Yet further object of the present invention is to provide for a method of detection and/or quantitation of polynucleotide sequences in sample of biological and/or non-biological material through target polynucleotide sequence amplification which can be carried out on polynucleotides that may be present in any biological or non-biological sample, such as clinical samples, for example blood, urine, sputum, saliva, faeces, pus, semen, serum, other tissue samples, culture media, fermentation broth and the like with or without pre-extraction or purification of analytes by known methods to concentrate nucleic acids.

Yet another object is to provide an improved method for detection and/or quantitation of polynucleotide sequence or sequences in a sample in very short time and in standard tube or 96 well microtitre plate/96 tube tray format so that large number of sequences can be detected or quantitated in short time, which can be useful for RNA expression profiling.

Yet another objective is to provide a method for high through put RNA expression profiling for large scale analysis of absolute quantities of mRNAs both in homogeneous phase as well as in heterogeneous phase by using amplification primers of many nucleic acid amplification reactions.

Another object is to detect the amplification product (of the size of primer dimer) by utilizing intercalating fluorescent dyes like ethidium bromide, picogreen, SYBER TMGREEN 1, acridine orange, thiazole orange, chromomycin A3 and YO-PRO-1 and other signal generation methods and other signal generation methods.

Yet further object of the present invention is to develop kits and labeled oligonucleotide amplification primer or primer-probe sets for the detection and/or measurement. of polynucleotide nucleic acid amplification products, polynucleotide nucleic acid target sequence in the sample which would favour effective and improved detection and quantitation of polynucleotide sequences in samples of biological and non-biological materials.

### SUMMARY OF THE INVENTION

Thus according to the basic aspect of the present invention there is provided a method of detection and/or quantification of target nucleic acid sequence by nucleic acid amplification reaction comprising:

MET/FRET between a donor moiety and an acceptor moiety provided separately on at least two separate oligonucleotides that are part of the opposite complementary strands of a nucleic acid segment with the donor and acceptor moieties separated from each other by 0-25 nucleotide pairs when the two labelled oligonucleotides are hybridized to and/or incorporated in the amplification product.

The above two labeled oligonucleotides can be used as two amplification primers or as one amplification primer and the other probe or as two probes and of linear or hair-pin configuration.

The two primers can be linear, or one linear and one hair-pin, or both hair-pin, the hair-pin one containing the acceptor moiety near the 3′ end (within 2-10 nucleotides away from 3′ end) and a quencher for the acceptor near the 5′ -end to quench the acceptor or both hair-pin each containing a donor or an acceptor moiety near the 3′ end (within 2-10 nucleotides away from 3′ end) and respective quencher or quenchers near the 5′-ends of both, quenchers being different from the donor and the acceptor moieties. When ever the hair-pin stem structure can quench the donor or acceptor providing quencher may be redundenco.

In accordance with one aspect of the invention, the method of detection of target nucleic acid sequence by nucleic acid amplification comprise a method of detection of target nucleic acid sequence by nucleic acid amplification reaction comprising (i) use of at least two oligonucleotides as a pair of primers for amplification of said target sequence; (ii) the 3′ ends of said pair of primers being on two opposite strands and separated from one another by 0-25 nucleotide pairs in the final amplification product; and (iii) carrying a denaturation step and atleast an annealing step in each cycle.

The two oligonucleotides are so designed that they bear a specific distance relationship between them so that they bring the donor and the acceptor moieties on them within the distance of 50% energy transfer between the donor and the acceptor when hybridized to and/or incorporated into the amplification product the donor and the acceptor MET moieties being in two opposite strands. Such distance relationship gives the additional specificity of detection. The size of the amplification product due to the 3′ ends of the primers being 0-25 nucleotide pairs away from one another make the PCR amplification efficient and increase the yield of the amplification product 8-10 times that of amplification products of other sizes.

Importantly, the above selective process is effective and achieves higher efficiency of amplification of an amplification product of the size close to that of the primer dimer for making the method more reliable and more sensitive through less PCR failure. In particular, primer dimer is identified as an efficient template for amplification and accordingly higher efficiency of amplification result in higher yield of the amplification product and which also reduce to large extent the formation of non-specific amplification product. The incorporation of the donor and the acceptor labeled amplification primers into the amplification product is directed to bring the donor and the acceptor moieties separated by 3-20 most preferably by 4-10 nucleotide pairs provided such labeled primers are incorporated into the amplification product as efficiently as unlabeled primers.

In the method of the invention. the amplification primers (forward and reverse) were developed for the amplification of a target segment of the size almost close to that of the primer dimer i.e., the length of the forward primer plus the length of the reverse primer plus zero to twenty-five bases. Both the primers were tested for not forming primer dimers particularly heterodimers. These primers were suitably labeled and formed a product when they contacted a target sequence. Additionally the amplification of above size product being very efficient less amount of primer will be required for the amplification reaction, chances of formation of primer dimer will reduce further.

In accordance with another aspect of the present invention the method for detection of target nucleic acid sequence by nucleic acid amplification comprise (i) use of two oligonucleotides as a pair of primers for amplification of said target sequence; (ii) the 3′ ends of said pair of primers being on two opposite strands and separated from one another by 0-25 nucleotide pairs in the final amplification product; and (iii) carrying out denaturation for a period less than 10 seconds, and annealing of less than 5 seconds and extension of 0 second in each cycle. Further carrying out the PCR amplification reaction for first 10-20 cycles with reduced annealing time and for the remaining cycles with little longer annealing time allows detection of the target with high specificity without reduction in the yield of the amplification product.

Further use of 0.1° C. per second ram rate of heating and cooling completes 30 cycles amplification reaction in about 50 minutes. By using higher ram rate of heating and cooling available with present day thermal cyclers and suitably choosing annealing time a 30 cycles amplification reaction can be completed in 20-30 minutes. This will allow very fast analysis of target nucleic acid. Moreover ordinary PCR amplification tube and 96 tube trays can be used.

Advantageously,in the method of the invention the fluorescence energy emitted by the donor moiety on excitation is absorbed by the acceptor moiety, which in turn releases the absorbed energy by emitting light at different wavelengths. The measurement of fluorescent emission from the acceptor gives the measure of the amplification reaction. Measurement of the reduction in donor fluorescence in addition to the acceptor fluorescence helps in counter checking the result.

Further, in addition to the above acceptor emission measurement, if the acceptor is excited with the acceptor specific radiation or light the increase in acceptor emission will give the measure of the total emission i.e., the emission from specific and non-specific product formation. This emission measurement can be easily normalized for excitation by the donor specific excitation radiation. Thus subtraction of this normalized acceptor emission from acceptor emission utilizing donor specific radiation will give the measurement of the MET, which will be the correct measurement of the specific amplification product.

The above method is advantageously found to be quantitative (less or no background), because measurement is based on sensitized emission by molecular energy transfer and not based on removal of fluorescence quenching used in prior art; and moreover non-specific signals due to non-specific incorporation of MET primer into the amplification product is not accounted in the signal. Further breakage of covalent linkages between the FRET moieties and the oligonucleotides due to PCR condition and separation of donor and acceptor moieties from the labeled oligonucleotides due to degradation of the labeled oligonucleotides by exonuclease activities of the polymerase also do not contribute to the signal. The fluorescence/emission background due to direct excitation of the acceptor moiety by the light used for donor excitation is reduced by suitably selecting acceptor whose excitation spectra overlap with emission spectra of the donor towards the longer wavelength end of the spectra maintaining a balance between background and energy transfer or using quencher to quench acceptor emission or using quenchers to quench both donor as well as acceptor emission in a hair-pin configuration or a configuration to serve the same purpose. Donor-acceptor pairs having spectral overlap around 25% would most suitable.

Further the acceptor, can be a non-radiative fluorophore, i.e. a quencher, which absorbs the energy emitted by the donor but does not emit any light. Decrease in the donor emission give the measure of the amplification process or the target sequence present in the sample. The quenching can be achieved by any known method. In particular the quenching can be attended following anyone of the methods:
(I) at least the oligonucleotide labeled with the acceptor is provided in a hair-pin quenched configuration, where the acceptor is provided quenched with a quencher or both the donor as well as the acceptor labeled oligonucleotides are provided in hair-pin quenched configuration so that both the donor and the acceptor moieties are provided quenched with two separate quenchers. The quenchers are attached at or near the respective 5′ ends, the quencher and the acceptor or the donor are on two opposite strands of the stem structure and part of the same oligonucleotide. In the event of formation of hair-pin stem structure the nucleotide to which the donor or the acceptor moiety is attached is complementary and opposite to the nucleotide to which the quencher is attached or the nucleotide to which the donor or the acceptor moiety is attached and the complement of the nucleotide to which the quencher is attached are within five nucleotides, the donor labeled and or acceptor labeled hair-pin quenched oligonucleotides remain quenched when not incorporated into or hybridized to the amplification product.
(II) using additional one or two oligonucleotides as the case may be each being labeled separately at or near 5′ end position with suitable quencher for the acceptor or the donor MET moiety such that one member of the quencher labeled additional oligonucleotide is fully or partly complementary to the acceptor labeled oligonucleotide resulting in quenching of the acceptor when the acceptor labeled oligonucleotide is not incorporated into or hybridized to the amplification product and the second member of the quencher labeled additional oligonucleotide is fully or partly complementary to the donor labeled oligonucleotide resulting in quenching of the donor when the donor labeled oligonucleotide is not incorporated into or hybridized to the amplification product; and
(III) by providing the acceptor labeled oligonucleotide linked to another suitable oligonucleotide complementary partly or fully to this acceptor labeled oligonucleotide and labeled with a quencher at or near its 5′ end through non-nucleotide organic linker or by providing both the acceptor and donor labeled oligonucleotides linked to two separate additional suitable oligonucleotides fully or partly complementary to the acceptor and donor labeled oligonucleotides through non-nucleotide organic linkers respectively and labeled at or near their 5′ ends with two quenchers respectively so that the quenchers can quench the acceptor and the donor when the acceptor and the donor labeled oligonucleotides are not incorporated into or hybridized to the amplification product.

The method efficiently utilizes another advantage of hair-pin primers, i.e., the hair-pin primers are efficient (a few times) over linear primers and gives better specificity of primer annealing. Because of higher efficiency of hair-pin primers smaller amount, i.e., lower concentration of primers would be required that would in turn further reduce primer dimer. formation in the amplification reaction. Moreover stable stem structures of hair-pin primers remain in closed configuration during annealing step in absence of target sequence thus preventing primer dimer formation further. Further method also utilize the advantage of hair-pin probe efficiency over linear probe that the reduce the conc. of probe required

Preferably, the hair-pin oligonucleotides of stem length of 8-9 nucleotides were found to provide stable stem structure of the oligonucleotides at 1.5 mm Mgcl₂ conc. Thus when such hair-pin oligonucleotides were used as amplification primers in PCR reaction there was no primer dimer formation even at higher primer concentration and there was no decrease in the yield of the amplification product due to any sluggishness in the opening of the stem structure.

In accordance with another aspect of the present invention there is provided a first oligonucleotide primer pair selected to amplify a first segment of the target nucleic acid used at appropriate concentration, and a second oligonucleotide amplification primer designed to amplify a second segment of the first segment the second oligonucleotide primer pair being suitably labeled for MET. Alternative a third oligonucleotide primer suitably labeled for MET in association with the above first member of first pair suitably labeled is nested with signal being generated on said selective amplification of the target nucleic acid.

The oligonucleotide primer pair selected to amplify a segment of the target nucleic acid is used at appropriate concentration where one of the said oligonucleotide primer pair is a first member of the labeled oligonucleotide primer pair, a third oligonucleotide suitably labeled for MET and designed to hybridize to the strand of the amplified segment into which the first member of the labeled oligonucleotide primer get incorporated into so as to facilitate detection of the amplification process and without displacement of the third oligonucleotide before measurement of the signal, the said oligonucleotide being complementary to the target sequence and not extended by the polymerase, both the labeled oligonucleotides being labeled suitably at or near their 3′ ends.

Advantageously, in the above process of the invention the amplification reaction comprise the steps of adding polymerase, reaction buffer, deoxy nucleoside triphosphates in addition to the effective amounts of the amplification primers to the samples, cycling the sample between at least a denaturation temperature and an elongation temperature, exciting the reaction mixture with the donor exciting radiation or light, measuring the emission of the acceptor MET moiety and optionally the reduction in donor emission, thus allowing detection of nucleic acid target without creating inhibition to amplification reaction and signal measurement without loss of signal.

In accordance with another aspect of the present invention there is provided a method of detection wherein
(a) atleast the oligonucleotide labeled with the acceptor is provided in quenched configuration such that the acceptor remains quenched when the acceptor labeled oligonucleotide is not incorporated into or not hybridized to the amplification product, thus reducing the background and remains unquenched in open configuration of the oligonucleotide producing signal when incorporated into or hybridized to the amplification product,
(b) the amplified sample is illuminated with light absorbed by the donor MET moiety, and
(c) monitoring the sensitized emission from the acceptor and optionally emission from donor of the MET pair moieties.

In accordance with another aspect the present invention provides a method wherein a first oligonucleotide of linear or hair-pin configuration labeled with a donor moiety at or near preferably near its 3′ end and a second oligonucleotide singly labeled at or near preferably near its 3′ end also with an acceptor moiety capable of absorbing the energy or light emitted by the donor, where the acceptor is selected from a fluorophore or a quencher preferably a quencher including DABCYL or its analogue or nanogold particle, black hole quencher, the donor moiety of the first oligonucleotide kept quenched when the first oligonucleotide is not incorporated into the amplification product either by providing a third oligonucleotide fully complementary to the first oligonucleotide seperately or linked to first oligonucleotide through an organic linker and labelled at or near its 5′ end with a quencher moiety or by providing the first donor labelled oligonucleotide as hair-pin oligonucleotide with a quencher at or near its 5′ ends so configured that the quencher comes in close proximity to the donor moiety in its stem structure, the quencher is selected to be capable of absorbing the energy or light emitted by the donor, and selected to be a fluorophore or a non-radiative quencher preferably a quencher including DABCYL or its analogue or nanogold particle or black hole quencher, the first and second oligonucleotides are the two primers of nucleic acid amplification reaction and are used such that the emission of the donor is quenched by the quencher/acceptor on the second oligonucleotide only in case of formation of primer dimer but in case of specific amplification product formation the above said quencher/acceptor of second oligonucleotide remains at least 10 bases away from the donor moiety incorporated into the amplification product through the first oligonucleotide and at the same time the quencher of first oligonucleotides remains at least 10 bases away from the donor moiety incorporated into the amplification product through the first oligonucleotides thus allowing the donor moiety to emit its characteristic energy or light and signal generation for the detection or quantitation of a target nucleic acid sequence with increased signal to noise.

The first oligonucleotide of the invention labeled at or near its 3′ end with the acceptor MET moiety of a donor-acceptor MET pair, and is of hair-pin configuration; in one strand of the stem structure of the hair-pin oligonucleotide the acceptor MET moiety is attached and in the complementary strand of the stem a quencher is attached; the acceptor and quencher are so configured that emission of the acceptor moiety remains maximum quenched in the closed configuration of the above hair-pin oligonucleotide. The above oligonucleotide is so labeled that when it is an amplification primer, the amplification reaction or the primer extension is not affected. Because of the acceptor MET moiety remaining quenched when the above said oligonucleotide remains free in solution without being incorporated into or hybridized to the amplification product, the excitation of the above said acceptor MET moiety by the donor excitation radiation/light is negligible resulting in low background emission from the acceptor MET moiety. On the other hand when the above said labeled hair-pin oligonucleotide is either incorporated into or hybridized to the amplification product remains in open configuration separating the acceptor MET moiety and the quencher. On illumination by donor excitation radiation or light the acceptor-MET moiety gets partially excited, emitting its characteristic emission. The ratio of this emitted energy (i.e. signal to the background) would be the same as that if it was illuminated with the radiation/light characteristic for acceptor excitation. Further during MET between the donor moiety on the second oligonucleotide, which is again a primer, the excitation energy of the donor due to excitation of same with the donor excitation radiation (light) is transferred to the acceptor MET moiety, which in turn emits absorbed energy as its characteristic emission, thus augmenting or increasing the signal (acceptor emission). Further when the second donor labeled oligonucleotide primer is also provided as a quenched primer by many of the ways known in the art the background will be still less when the primer is not incorporated into the amplification product but when incorporated into the amplification product there will be seperation of the quencher from the donor moiety thus allowing emission from the donor moiety a part of which will contribute to the measured emission in the characteristics emission range of the acceptor thus resulting in further lowering of the background and enhancement of the signal. As a whole the signal will have a component from acceptor emission due to excitation of acceptor by donor excitation radiation, a component from donor emission in the emission measurement range of the acceptor and a FRET component from the energy transfer from the donor to the acceptor and finally higher signal from higher yield of the amplification product. All together the signal to background/noise ratio increase to a large extent resulting in higher sensitivity of detection. Further digestion of the hair-pin quenched probe and/or primer does not contribute to the noise as much as it does in case of use of hair-pin quenched primers and probes in the other methods known in the art.

According to another aspect the method of detection of target nucleic acid sequence comprise quenching the donor and/or the acceptor moieties on the oligonucleotide preferably with non-radiative quencher or quenchers which absorb, light in the entire visible region or the spectral emission region of the donor and/or the acceptor, and which is part of a second oligonucleotide partly or fully complementary to the target sequence and fully complementary to the last five to nine bases at or near the 3′ end of the primer, and is attached to the 5′ end of the above said primer through a short linker at the 3′ end of the above said second oligonucleotide. The above said second oligonucleotide is so designed that the labeled probe or labeled primers remain quenched when not hybridized to or not incorporated into the amplification product and remains in open configuration when the above probes/primers hybridize to or get incorporated into the amplification product. The above linker is either a third oligonucleotide of length between two bases to twelve bases, which may or may not be fully or partly complementary to the target sequence or a short organic non-nucleotide linker or linker and spacer. The MET moieties are placed near the 3′ end of the oligonucleotide primer and the quencher (like DABCYL or its derivatives or like which absorbs in the entire visible region or any other quencher which absorbs in the spectral emission region of the donor and /or the acceptor MET moiety) is placed at or near the 5′ end of the second oligonucleotide; in such a way that the donor and/or the acceptor MET moieties are in close proximity to the quencher in order that quenching of emission from the donor and/ or acceptor can take place in closed configuration, i.e, when the oligonucleotides are not incorporated into or hybridized to the amplification product. MET between the donor and the acceptor moieties occurs when the two labeled oligos get incorporated into or hybridized to the specific amplification product or one gets incorporated into and the other hybridized the amplification product. The emission of the acceptor MET moiety and optionally that of the donor is measured to monitor the amplification process or detection and/or quantitation of the amplification product.

In still another embodiment the donor and/ or the acceptor moieties on the oligonucleotide primers are provided quenched with the help of additional one or two more oligonucleotides labeled at or near their 5′ ends with suitable quencher for donor and/ or acceptor respectively and complementary partly or fully to the oligonucleotide primers, when not incorporated into the amplification product. The signals are generated on illumination by donor specific excitation radiation and from the separation of the donor and acceptor labeled oligonucleotide primers from the quencher labeled complementary oligonucleotides as the above donor and acceptor labeled primers are incorporated into the amplification product and MET takes place. In case of use of labeled oligonucleotide as probe the donor or acceptor moiety on the probe is provided quenched with the help of an additional oligonucleotide complementary partly or fully to the probe and labeled at or near its 5′ end with quencher. The quencher can be radiative quencher or non-radiative quencher

The energy transfer efficiency kₑₜ is given by the expression kₑₜ=8.79×10⁻²⁵κ²φ_{D}K_{D}η⁻⁴R⁻⁶J, where R is critical distance for 100% energy transfer, J is the overlap integral between the two spectra. Reduced J and reduced R will give same kₑₜ value. Choosing an acceptor overlapping the donor spectra near the longer wavelength end and placed close to donor will give reasonably good energy transfer but with reduced excitation with light used for exciting the donor, i.e. reduced background. An acceptor with high quantum yield and high extinction coefficient of absorption is an acceptor of choice. Similarly a donor of high extinction coefficient of absorption and high quantum yield is a donor of choice. A spectral overlap around 25% is desirable for balance between background and energy transfer.

In another preferred embodiment at least the acceptor moiety or both the donor and the acceptor moieties on the primers/probe are quenched with a quencher like DABCYL, or other suitable quencher where the primers/probe are labeled separately at or near their 3′ ends preferably near their 3′ ends, with the donor or the acceptor moiety and the MET moieties on the labeled oligonucleotide primers are kept quenched when not incorporated into or hybridized to the amplification product with the help of another two additional 3′ end capped oligonucleotides as such or suitably labeled with quenchers at or near their 5′ ends.

In another embodiment two oligonucleotides amplification primers both linear and one of them is suitably labeled either with a donor MET moiety or an acceptor MET moiety at or near 3′ end preferably near 3′ end (with in 2-10 nucleotides away from 3′ end) are used. On amplification of the target sequence both the primers get incorporated into the two opposite strands of the amplification product. A third oligonucleotide linear and suitably labeled either with an acceptor MET moiety or a donor MET moiety respectively at or near 3′ end is provided so that this labeled third oligonucleotide hybridize to the strand of the amplification product into which the first labeled oligonucleotide primer got incorporated thus bringing the donor MET moiety and the acceptor MET moiety within FRET/MET distance. The donor moiety is excited by its characteristic excitation light or radiation and the emission of the acceptor moiety and optionally reduction in that of the donor moiety is measured. When the acceptor is a non-irradiative acceptor that is a quencher there will be no emission from the acceptor and the reduction in the emission of the donor moiety is measured.

In a further embodiment, the invention provides two or four linear and/or hair-pin oligonucleotides primers (non-duplex), which are separately labeled with donor or acceptor MET moieties such that MET will occur only when the respective primers are ligated in Ligase Chain Reaction (LCR). In this embodiment the oligonucleotides are in hair-pin configuration, where near the one end of the stem there is a donor or an acceptor MET moiety attached and at the other end opposite to the MET pair moiety there is a non-radiative quencher like DABCYL, so that the emission of the MET moiety of the unligated oligonucleotides are quenched. On ligation of the oligonucleotides there is MET between the donor and acceptor moiety. In case of four labeled oligonucleotide there will be both intrastrand and interstrand FRET/MET between the donor and the acceptor moieties and the energy transfer will be almost complete, thus resulting in enhanced signal and higher signal to noise ratio.

The oligonucleotides for use in the invention can be of any suitable size, preferably in the range of 10 to 40 bases, and more preferably between 15 to 30 bases. The oligonucleotide can be DNA or RNA or chimeric mixtures or derivatives or modified versions there of, so long as it is capable of priming the amplification reactions or hybridizing the desired amplification product. In addition to being labeled with a MET moiety, the oligonucleotide can be modified at the base moiety, sugar moiety or phosphate backbone, and may include other appending groups including linker or spacer arms, or labels so long as it is still capable of priming the amplification reaction or hybridizing with the amplification product as a probe.

For example the oligonucleotide may comprise at least one modified base moiety which is selected from the group including but not limited to 5-bromo-uracil, 5, fluoro-uracil, 5-chloro-uracil, 5-iodo-uracil, hypoxanthine, xanthine, 4-acetylcytosine, 5-(carboxy hydroxy methyl) uracil, 5-carboxymethyl aminomethyl-2 thiouridine, 5′-carboxy methyl aminomethyl uracil, dihydrouracil, b-D-guanosine, inosine, N6-isopentynyladenine, 1-methyl guanine, 1-methyl inosine, 2,2-dimethylguanine, 2-methyl adenine, 2-methyl guanine, 3-methyl cytosine, 5-methyl cytosine, N6-adenosine, 7-methyl guanine, 5-methyl aminomethyl uracil, 5-methoxy aminomethyl-2 thio uracil, b-D-mannosyl queosine, 5-methoxy carboxymethyl uracil, 5-methoxy uracil, 2-methyl thio-N6-isopentenyladenine, uracil 5-oxyacetic acid (v), wybutoxosine, pseudouracil, queosine, 2-thiocytosine, 5-methyl-2-thiouracil, 2-thio-uracil, 4-thiouracil, 5-methyl uracil, uracil-5-methyl ester, uracil 5-oxyacetic acid (v) 5′ methyl-2-thiouracil, 3-(3-amino-3-N-2-carboxypropyl) uracil, (acp)3W, and 2,6 diaminopurine. The oligonucleotide may also comprise at least one modified sugar moiety selected from the group including but not limited to arabinase, 2-fluoroarabinose, xylulose and hexose.

In addition, the oligonucleotide may comprise at least one modified phosphate backbone selected from the group consisting of a phosphorothioate, a phosphorodithioate, a phosphoramidothioate, a phosphoramidate, a phosphoradiamidate, a methyl phosphonate, an alkyl phosphotriester, formacetal, peptide nucleic acid or analong thereof

Oligonucleotides of the invention may be synthesized by standard methods known in the art, for e.g., by de novo chemical synthesis using an automated DNA synthesizer (such as commercially available machines from Biosearch, Applied Biosystems and many other suppliers using phosphotriester chemistry) or by cleavage of a larger nucleic acid fragment using non-specific nucleic acid cleaving chemicals or enzymes or site specific restriction endonucleases. Alternatively those can be obtained from commercial suppliers.

Phosphorothioate oligonucleotides may be synthesized by the method of Stein et.al Nucl. Acids Res. (1988, 16, 3209), methylphosphonate oligonucleotides can be synthesized by the method of Sarin et.al. (Proc. Natl. Acad. Sci. USA 1988, 85, 7448-7451) etc.

The oligonucleotides can be purified by any method known in the art, including extraction, gel permeation chromatography, gel electrophoresis and HPLC purification. The concentration of the oligonucleotide can be measured by measuring optical density at 260 nm in a spectrophotometer. Purity of the oligonucleotide can be determined by polyacrylamide gel electrophoresis or HPLC as known in the art.

The oligonucleotides of the invention may be labeled with the donor and acceptor moieties as well as the quencher during chemical synthesis or by attachment after synthesis by methods known in the art. Both the donor and the acceptor moieties are fluorophores, europium and terbium chelates, quenchers or other entities. Suitable moieties that can be selected as donor or acceptor in FRET pairs, as well as the quencher moieties are given in the table. Selection of the donor and acceptor of FRET pair is decided on the basis of the spectral overlap of the two fluorophores and molar extinction coefficient of absorption of radiation or light and quantum yield as known in the art.

TABLE
Suitable moities that can be selected as donor or acceptors in FRET pairs.

4-acetamido-4′-isothiocyanatostilbene-2,2′ disulfonic acid
acridine
acridine isothiocyanate
5-(2′-aminoethyl) aminonaphthalene-1-sulfonic acid (EDANS)
4-amino-N-3-vinylsulfonyl) phenylnaphthalimide-
3,5 disulfonate(lucifer yellow vs)
N-(1-anilion-1-naphthyl) maleimide
anthranilamide
brilliant yellow
coumarin
7-amino-4-methylcoumarin (amc, coumarin 120)
7-amino-4-trifluoromethylcoumarin (coumarin 151)
cyanosine
cyanine-3
cyanine-5
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-dimethylamino1 Naphthalene-1-sulfonylchloride (DNS, dansyl chloride)
4-(4′-dimethylaminophenylazo) benzoic acid (DABCYL)
4-dimethylaminophenylazophenyl-4′-isothiocyanate (DABITC)
eosin
eosin isothiocyanate
erythrosin and derivatives;
erythrosine b
erythrosine isothiocyanate
ethidium
fluorescein
fluorescein isothiocyanate
5-carboxyfluorescein (5-FAM)
6-carboxyfluorescein (6-FAM)
5-(4,6-dichlorotriazin-2-yl) aminofluorescein (DTAF)
2′7′-dimethoxy-4′5-dichloro-6-carboxyfluorescein (JOE)
fluorescamine
IR144
IR1446
Malachite green isothiocyanate
4-methylumbelliferone
ortho cresolphthalein
nitrotyrosine
pararosaniline
Phenol Red
B-phycoerythrin
pyrene
pyrene butyrate
succinimidyl 1 pyrene butyrate
Reactive Red 4 (cibacron .RTM .Brilliant Red 3B-A)
6-carboxy-X-rhodamine (ROX)
6-carboxyrhodamine (R6G)
lissamine rhodamine B sufonyl chloride
rhodamine(Rhod)
rhodamine B
rhodamine 123
rhodamine x isothyiocyanate
sulforhodamine b
sulforhodamine 101
sulfonyl chloride derivative of sulforhodamine 101
(Texas Red)
N,N,N′,N′-tetramethyl-6-carboxyrhodimne (TAMRA)
tetramethyl rhodamine
tetramethyl rhodamine isothiocyanate(TRITC)
riboflavin
rosolic acid
terbium chelate derivatives
europium chelate derivatives

In the above method of the invention, the oligonucleotides used are preferably selected to have the following sequences:

| | |
|---|---|
| 1). | 5′-GGG GTA CTA CAG CGC CCT GA -3′ |
| 2). | 5′-GGG GTA CTA CAG CGC CCT GA -3′ |
| | \| |
| | FAM |
| 3). | 5′-GTC CTG GAA GAT GGC CAT GG -3′ |
| 4). | 5′-GTC CTG GAA GAT GGC CAT GG -3′ |
| | \| |
| | JOE |
| 5). | 5′-ATG GCC ATC GTC CTG GAA GAT GGC CAT GG -3′ |
| 6). | 5′-DABCYL-ATG GCC ATC GTC CTG GAA GAT GGC CAT GG -3′ |
| | \| |
| | JOE |
| 7). | 5′-DABCYL-ATG GCC ATC GTC CTG GAA GAT GGC CAT GG -3′ |
| | \| |
| | FAM |
| 8). | 5′-GCT CAT GGC GCC TGC CTG G -3′ |
| | \| |
| | DABCYL |
| 9). | 5′-ATG GCC ATG TCC TGG AAG ATG GCC ATG G -3′ |
| 10). | 5′-GGG GTA CTA CAG CGC CCT -3′ |
| | \| |
| | FAM |
| 11). | 5′-GTC CTG GAA GAT GGC CAT GG -3′ |
| | \| |
| | Rhod |
| 12). | 5′-GTC CTG GAA GAT GGC CAT GG -3′ |
| | \| |
| | JOE |

According to another aspect the method of detection of target nucleic acid sequence comprise a kit for use in method of analogous detection and/or quantitation of target nucleic acid sequence or sequences present in a sample comprising
(a) a polymerase or polymerases
(b) atleast a first oligonucleotide of sequence complementary to the nucleotide sequence flanking a target nucleotide sequence suitably labeled with a donor MET/FRET moiety at or near 3′ end.
(c) atleast a second oligonucleotide of sequence at 5′ end of the first nucleotide sequence complementary to the nucleotide sequence flanking the target nucleotide sequence or the segment of the target nucleotide sequence suitably labeled with an acceptor MET/FRET moiety at or near 3′ end.
(d) deoxy nucleotides in solution (water or buffer) or lyophilized.
(e) a reaction buffer for the nucleic acid amplification reaction.
   wherein the first and the second oligonucleotide sequences comprise the two primers (forward and reverse) of many nucleic acid amplification reactions and adapted to generate a detectable signal if the two oligonucleotides get incorporated into two opposite strands of amplified product and come in right proximity. In further extension of this kit a third oligonucleotide suitably labeled at or near 3′ end, which is a probe is provided. Further the oligonucleotides of the kit can be linear labeled oligonucleotide or any quenched labeled oligonucleotide of this invention.

The above kit of the invention may be research kits, diagnostic kits or otherwise, where the nucleic acid target being amplified is correlated with the presence or absence of a disease or disorder of human, plant or otherwise, presence or absence of an infectious agent of human, plant or otherwise, presence or absence of specific genetic trait or marker of human, plant or otherwise and absolute quantities of expressed and global absolute quantitation of large number of expressed RNAs.

The method of detection of target nucleic acid sequence of the invention achieves the detection and/or quantitation of polynucleotide sequence or sequences in a sample in very short time and in standard tube or 96 well microtitre plate/96 tube tray format so that large number of sequences can be detected or quantitated in short time, which can be useful for RNA expression profiling.

In accordance with another aspect of the invention there is provided a high through put homogeneous phase nucleic acid amplification assay for real time quantitative RNA expression profiling. For real time quantitative RNA expression profiling, mRNAs in a sample are converted to c-DNAs by any one of the known methods in the art and an additional sequence is attached to the 5′ end of all c-DNA molecules in the sample by known methods in the art. Two primers are selected for amplification of a product with in the size range of the size of the first primer plus the size of the second primer plus 0-25 bases. A first primer is designed from the additional sequence attached to the c-DNA and a second primer is designed from the 5′ end of a c-DNA. The first primer is common for amplification analysis of all c-DNAs, while the second primer is specific for its c-DNA/mRNA, i.e the first primer is a common universal primer for all c-DNAs and the second primers are different and specific for each c-DNA /mRNA. For measurement or quantitation either the universal first primer is provided un labeled and the second primers (c-DNA specific) are labeled with a donor MET moiety maintained quenched in its unincorporated state providing an acceptor MET mmoiety by different means of quenching known in the art or the universal first oligonucleotide primer is labeled with a donor moiety and the second c-DNA/mRNA specific primers are labeled with acceptor MET moiety or the universal first oligonucleotide primer is labeled with an acceptor fluorophore MET moiety or a non-radiative quencher near its 3′ end and the second c-DNA/mRNA specific primers are labeled near their 3-ends with different donor MET moieties, so that on amplification of specific c-DNA or RNA there is increase or decrease in the emission of a specific donor moiety or increase in emission of a specific acceptor moiety. By this method of measurement a large number of mRNAs can be quantitated in a single amplification reaction. The number of mRNAs/c-DNAs that can be quantitated will depend on instrumentation.

In still further embodiment the invention provides a high through put homogeneous phase nucleic acid amplification assay for real time quantitative RNA expression profiling in which first mRNAs in a sample are converted to c-DNAS and an additional sequence is attached to the 5′ end of all c-DNA molecules by known methods in the art. Next c-DNAs are digested with restriction enzyme preferably a four based cutter and a second additional sequence is ligated to the restriction digested sites of the c-DNAs. A first universal primer is selected from any one of the two additional sequences and a second specific primer for each c-DNA is selected form the region of the c-DNA adjacent to the ligated additional sequence from which the first primer was selected. For measurement either the first universal primer is provided unlabeled and the second primer is provided labeled with a donor MET moiety and an acceptor MET moiety and so labeled that the donor remains quenched when the primer is not incorporated into the amplification product, and the signal is generated after carrying out amplification reaction when the labeled second primer is incorporated into the amplification product and the reaction mixture is irradiated with donor specific excitation radiation; or the first universal primer is provided labeled near its 3′ end with a donor moiety or an acceptor MET moiety (preferably a non-radiative quencher) and the second specific primers are labeled respectively with an acceptor MET moiety (radiative, i.e fluorophore) are donor MET moiety a signal is generated by contacting the sample, carrying out the amplification reaction and irradiating with donor excitation radiation and measuring acceptor emission or donor emission quenching. Number of mRNAs/c-DNAs that can be quantitated in a single reaction will depend on instrumentation and the donor-acceptor moiety selection. In further extension of this embodiment is provided a first universal primer selected from the first ligated additional sequence and a second specific primer as selected from specific mRNA/c-DNA and a third universal primer selected from the second ligated additional sequence all three primers are nested in a nested PCR reaction and is used in high through put RNA expression profiling. And still further extension of the embodiment the second specific primer is used as a probe instead of a primer and is used in high through put RNA expression profiling.

In still further embodiment the invention provides a high through put homogeneous phase nucleic acid amplification assay for real time quantitative RNA expression profiling using nucleic acid sequence based amplification. First al mRNAs in the sample are converted to c-DNAs, then an additional universal sequence carrying a T7 promoter sequence at its 5′ end is ligated to the 5′ ends of all c-DNAs and then carrying out nucleic acid sequence based amplification reaction on the sample as known in the art employing an universal primer for amplification of all c-DNAs/mRNAs selected from the above additional sequence in conjunction with specific primers selected from 5′ end regions of individual c-DNAs/mRNAs as required by the method of the invention. The primers are again suitably labeled as required by the method of the invention

The method for high through put RNA expression profiling for large scale analysis of absolute quantities of mRNAs can be carried out both in homogeneous phase as well as in heterogeneous phase by using amplification primers of many nucleic acid amplification reactions.

In further embodiment, the invention provides heterogeneous phase nucleic acid amplification (PCR/LCR) assay in addition to the homogeneous phase assay. In heterogeneous phase assay one of the amplification primers (labeled or unlabeled) is fixed or attached at its 5′ end to a non-porous solid support through a linker/spacer (preferably water soluble or hydrophilic) while the other primer (labeled or unlabeled) or the other amplification primers respectively, are in solution phase in contact with the non-porous solid support (glass, silicon wafer, polypropylene, polystyrene, and others preferable glass or silicon wafer) along with the other reagents required for the amplification. The solid surface can be flat like a glass slide or plastic laminate, and the like; or curved like a thin walled plastic tube or cuvette, a well or a microtiter plate or a silicon wafer microtiter plate and the like. In further extension of this embodiment the invention provides a heterogeneous phase nucleic acid amplification assay in addition to the homogeneous phase assay for large scale high through put real time quantitation of mRNAs/c-DNAs in a sample for quantitative RNA expression profiling which can be carried out for amplification of many targets using a common universal primer which remain in solution and while the individual specific primers for individual mRNA targets are fixed to solid surface through a linker and spacer (hydrophilic) in a manner analogus to the previous three embodiments. In still further of this embodiment tethered probes for individual mRNAs can also be used.

The above method of detection of target nucleic acid sequence to detect the amplification product (of the size of primer dimer) can also be carried out utilizing intercalating fluorescent dyes like ethidium bromide, picogreen, SYBER TMGREEN l,acridine orange, thiazole orange, chromomycin A3 and YO-PRO-1 and other signal generation methods.

The method provides a lower background and higher increased signal to noise ratio and accurate quantitation of the amplification product or the target sequence and the use of the same can be made in different methods of polynucleotide amplification including PCR, RT-PCR, NASBA, Ligase chain reaction, Strand displacement amplification (SDA), Triamplification.

Further the method is also applicable for detection of single nucleotide polymorphism, deletion and addition mutations, heterozygous mutations by denaturation profiling, repeat length mutations of small repeat, methylated DNA, and DNA polymorphism.

Analytes to be detected by the detection method of this invention are polynucleotides, which may be present in any biological or non-biological sample, such as clinical samples, for example blood, urine, sputum, saliva, feces, pus, semen, serum, other tissue samples, culture media, fermentation broth and the like. If necessary the analyte may be pre-extracted or purified by known methods of nucleic acid purification and extraction.

The pair of primers, i.e. one forward primer and one reverse primer, for use in PCR or RT-PCR or other PCR and nucleic acid amplification reactions consists of oligonucleotide primers that are complementary to the two different complementary nucleic acid strands of the target nucleic acid, such that the extension product of one primer towards the other primer generated by nucleic acid polymerase, can serve as template for the extension of the other primer. The nucleic acid amplification product is the content of nucleic acid in the sample between and including the two primer sequences. Nucleic acids that are “complementary” can be perfectly or imperfectly complementary, as long as the desired property resulting from complementarities, i.e. ability to hybridize is not lost.

In a specific embodiment, in the above method for the detection and quantitation of a nucleic acid amplification product in amplification reaction prior to said steps of amplification reaction the a sample comprising nucleic acid with two oligonucleotide primers, said oligonucleotide primers being adapted for use in said amplification reaction such that the said primers are incorporated into an amplified product of said amplification reaction, when a preselected target sequence is present in the sample; both the primers are individually labeled with either a donor moiety or an acceptor moiety in a way such that amplification reaction or primer extension can take place, where the acceptor moiety emits energy at one or more wavelengths different from that of the donor or as heat as the case may be. The present invention thus provides a method for the direct detection of the amplification product with improved sensitivity of detection maintaining a high specificity. It permits detection of amplification product without any separation, hence permitting detection without opening the tube, i.e. in close tube format thus reducing greatly the crossover contamination problem with amplification product that has slowed down the acceptance of PCR for routine analyses. The close tube format and the size of amplification product of the invention also enable high throughput of sample analysis and automation. The method gives higher signal to noise ratio and reduced PCR failure. The present invention also relates to kits for the detection and or measurement of nucleic acid amplification product, or products or detection and/or measurement of nucleic said target sequence or sequences.

Details of the invention, its objects and advantages are explained hereunder in greater detail in relation to the non-limiting exemplary illustration of the method of the invention:

Materials and Method:

Oligodeoxynucleotides Sequence ID Nos. 1 to 28 complementary to a 70 base pair synthetic target sequence and 600 bp segment of Leishmania donovani gp 63 gene were chemically synthesized on an Applied Biosystem oligosysnthesizer.

General Methods Followed:

1. All oligodeoxynucleotides (primers) were synthesized chemically by standard solid phase phosphotriester chemistry.

2. Single fluorophore labeling of the oligodeoxynucleotide primers.

The single labeling of the oligonucleotide primers with fluorophore at or near 3′ end was done through incorporation of a primary amino group by incorporating amino modified T-base (amino modified C₆dT) during synthesis as described by Ju et al (Proc. Natl. Acad. Sci. USA, 1995, 92, 9347-9351) and subsequent incorporation of fluorescent dyes into designated position of the oligonucleotides. Syntheized oligonucleotides were desalted and FAM (as donor) and JOE and Rhodamine (as an acceptor) were attached to a modified thymidine residue of the forward and reverse primers. Labeled oligonucleotides were purified by HPLC. Internal single fluorophore labeled oligonucleotide are available commercially.

3. Fluorophore and quencher double labeling of hair-pin oligodeoxy nucleotide primers:

The labeling of the hair-pin oligodeoxynucleotide primers with fluorophore near 3′ end and quencher at 5′ end are done through incorporation of a primary amino group by incorporating amino modified T base (amino modified C₆dT) during synthesis as described by Ju et al (Proc. Natl. Acad. Sci. USA, 1995, 92, 9347-9351) and incorporation of a thiol group at the 5′ end during synthesis using thiol phosphoramidite. After desalting the oligodeoxynucleotides were reacted to N-hydroxy succinamide derivative of the fluorophore, purified by HPLC and are subsequently reacted to N-(2 iodoethyl) trifluoroacetamide, desalted and reacted to DABCYL N-hydroxy succinamide (similar to fluorophore labeling of the above oligonucleotide; PNAS 1995, 92, 9347-9351).

Fluorophore and quencher double labeling of hair-pin oligodeoxynucleotide primers can be carried out incorporating suitable fluorophore dT phosphoramidite or Amino C6dT phosphoramidite for internal labeling with fluorophore for which phosphoramidite are not availlable and incorporatingDABCYL dT phosphoramidite at or near 5′ end during chemical synthesis of oligonucleotide and purifying by HPLC.

4. HPLC purification of oligonucleotide:

5′ DABCYL and 3′ fluorophore labeled oligonucleotides were purified by HPLC on C-18 reverse phase column using linear gradient of 0.1M triethyl ammonium acetate pH 6.5 and 0.1M triethylammonium acetate in 75% acetonitrile pH 6.5. There are many methods available for the same in the art.

5. Measurement of Energy transfer or FRET:—

Fluorescent resonance energy transfer (FRET) measurements were made in a Hitachi F4010 fluorescence spectrophotometer. Excitation wavelength was 488 nm and the emission spectra and measurements were taken between 500 nm and 600 nm.

6. Preparation of Leishmania donovani chromosomal DNA:

Leishmania donovani carrying cells were washed in PBS twice and pelleted at 3K, 10 min at 24° C. Cells were then resuspended in appropriate volume of Lysis buffer (150 mM NaCl, 10 mM EDTA, 10 mM Tris-HCl pH 7.5, 40 □l of 10% SDS per ml of buffer, 200 □g/ml Proteinase K) in a 15 ml Falcon tube. The tube was vortexed hard and incubated at 37° C. overnight or until the cell pellet dissolved. Phenol extraction was carried out with equal volume Tris equilibrated Phenol. The resulting suspension was centrifuged in microfuge at 3K for 10 min at RT. The DNA was precipitated with ethnol and was dissolved in water.

8. Fluorescence measurement

A Hitachi F 4010 Fluorescence spectrophotometer was used to measure the fluorescence spectra and fluorescence of the individual samples. 20 □l reaction mixture was diluted to 1000 □ with water and placed into a 1.0 ml cuvette at a temperature of 37 to 40° C. For the FAM/JOE FRET pair 488 nm excitation wavelength was used for excitation of FAM and the spectrum of JOE was measured between 500 and 600 nm.

9. Spectroscopic properties and sequences of linear oligonucleotides and estimation of FRET distance:

The sequence of the oligonucleotides and the location of the fluorescent labels on them are given in ‘Materials and Methods’. The donor FAM labeled forward primer Seq. no.5 consists of 20 nucleotides and carries the FAM label at base position 18. The reverse primer Seq. no.2 consists of 20 nucleotides. Acceptor JOE labeled oligonucleotide probes were labeled at their 3′ end with the fluorophore JOE.

10. Monitoring of PCR by sensitized emission and estimation of optimum FRET distance: To demonstrate that sensitized emission could be used to monitor PCR amplification reaction or amplification product, the synthetic template (Sequence given in FIG. 8) was amplified using the FAM labeled forward primer (Sequence no.5) and the reverse primer (Sequence no 2) in the presence of 3′ JOE labeled probes which bring the JOE label at distances of 5, 10, 15, 20 bases from FAM label in amplified product. After PCR amplification the tube was denatured and annealed once more and the amplified product was measured by illuminating the reaction mixture with FAM excitation wavelength of 488 nm light and measuring the emission of JOE at 553 nm at 37-40° C. There was a decrease in FAM emission (i.e., quenching of donor fluorescence) and increase in JOE emission. Energy transfer was observed upto the distance of 20 base pair and maximum energy transfer was observed at a distance of 5 base pair. The JOE labeled probes are not shown.

PCR Condition for Amplification

Amplification of the synthetic 60 bp target was performed in 100 □l volume of 20 nM Tris-HCl (pH-8.3), 50 mM KCl, 1.5 mM MgCl₂, 200 mM each dNTP, 400-500 nM each of the upstream primers, 0.01% gelatin, 3.0 units of Taq DNA polymerase, 1-5 ng of synthetic target sequence and thermal cycling of 2 minutes initial denaturation followed by 30 secs.

Denaturation at 94° C., 1 min annealing at 50° C. and 30 secs. extension at 72° C., 30 cycle and final extension at 72° C. for 2 mins.

11 Design of fluorophore labeled oligonucleotide primers:

General consideration of the primer design is same as that of Ex.1 E. Further a primer pair of high stringency can be labeled at or a few nucleotides away (preferably 2-10) from the 3′ ends while primer pair of low stringency should be labeled at a few nucleotides away (preferably 2-4) from 3′ ends.

### EX-1

### The Detection and/or Quantitation of a Nucleic Acid Target Using Target Amplication

### EXAMPLE: 1

(A) This example was carried out to demonstrate that in presence of template DNA specific amplification product only formed and no primer dimer formed.

In order to show that for amplification of an amplification product of the size close to that of primer dimer it is the specific amplification product and not the primer dimer that is formed during amplification reaction in presence of target DNA, primers were designed for the amplification of the 60 base pair segment (base Nos. 1094-1153) and 40 base pair segment (base Nos. 1114-11153) of gp63 gene of Leishmania donovani (accession No.M60048). The primer sequence for the study comprise seq. no.6, seq.no.8, seq. no.10, seq. no.13, seq. no. 16 and seq. no.17.

A. PCR condition for amplification of Leishmania donovani gp63 target sequence:

Amplification of gp63 target sequence of Leishmania donovani was performed in 25 □l volume of 20 mM Tris HCl pH-8.3, 50 mM KCl, 1.5 mM MgCl₂, 200 □M each dNTP, 200-400 nM each of the upstream and downstream primers, 0.01% gelatin, 3.0 units of Taq DNA polymerase, 100 ng/50 ng of chromosomal DNA and thermal cycling of 4 mins initial denaturation, followed by 30 seconds denaturation at 94° C., 1 min annealing at 60° C. and 30 seconds extension at 72° C., 30 cycles and final extension at 72° C. for 7 mins.

The PCR product formation was checked in 4% agarose gel run in 1XTAE buffer and the PCR product was quantitated by using ³²P or ³³P labeled dNTP and separating the labeled amplification product from unincorporated dNTPs in 10-20% non-denaturing polyacrylamide gel electrophoresis and gels were scanned in a Fuji Model Fuji film BAS-1800 Phosphorimager.

As appearing from FIG. 20 only specific amplification products and no primer dimer was formed, which indicated that amplification of an amplification product of the size close to that of primer dimer may be as efficient as the formation of primer dimer.

Further primers Seq. id no.6 and Seq. id no.16 were selected to give an amplification product of the size 47 bp. The same primer pair results in primer dimer formation at annealing temperatures between 55-65° C. But in presence of template give mostly amplification product and very little primer dimer. This is apparent from FIG. 21A lanes 1 and 2.

### Example: 2

This Example go to demonstrate the use of an amplification product of the size close to that of primer dimer for nucleic acid amplification helps in eliminating or reducing non-specific amplification product formation.

A number of amplification primers designed from an approximately 593 bp base pair segment (base nos.560-1153) of gp-63 gene of Leishmania donovani were used to amplify different segments of the same in the size ranges of 36 bp to 60 bp and 544 bp to 588 bp. In case of amplification of the amplification products in the size range of 36 to 60 bp there were no product formation other than the specific product i.e. no non-specific product formation of larger size (FIG. 21) while in case of amplification of the amplification products in the size range of 544-588 bp there was a lot of non-specific product formation (FIG.22, an annealing temperature of 60° C. was used). The amplification products in the size range of 36-66 bp were of the size, which was either the size of the forward primer plus the size of the reverse primer or, the size of the forward primer plus the size of the reverse primer plus 25 bases. This demonstrated that use of amplification primers to amplify a segment of target sequence of the size close to that of primer dimer (length of forward primer plus length of reverse primer plus zero to twenty five bases) could help in reducing largely or eliminating any non-specific product formation provided that the primers are designed and tested for no primer dimer formation. The same may be the result with an amplification product of size, size of the forward primer plus the size of the reverse primer minus two to three bases. Oligonucleotide sequences bearing the sequence ID Nos.6, 7, 8, 9, 10, 13, 14 and 15 were used in different combinations for amplifying different segment lengths of the above. In FIG. 21 in case of primer pairs seq. id nos.6&7, 8&9, 10&13 and 8&9 used for amplification of segments of L. donovani gp63 gene only in case of seq. id nos. 8&9 little non-specific product formation took place because of using annealing temperature of 58° C. and wide difference in melting temp. of primers seq no.8 and seq. id no.9. Primer seq nos.8 & 9 being hair-pin primers, when instead of hair-pin primers linear primers Seq. id no.17 & 18 (melting temperatures 66° C. and 62° C.) were used no non-specific product formation was observed (FIG. 21 A lanes 8 and 9 from left). Leishmania donovani genome target was chosen as this genome is highly GC rich and the target has 70% GC content, and hence most likely to result in lot of non-specific amplification product formation.

It was also observed that in case of amplification product of the size close to that of primer dimer even lower stringency of annealing did not result in any non-specific product formation whenever checked and gave consistently higher yield of amplification product. This supports that there will be less PCR failure in case amplification product of the size close to that of primer dimer is used for analysis, which would result in higher sensitivity of detection. Further in this case also higher amount of enzyme 3 units/100 μl PCR reaction was used through ordinary micro pipette and ordinary tip. This demonstrate that use of higher amount of enzyme by any means will affect amplification of a larger product by forming non-specific product than that of a smaller product of the size close to that of primer dimer.

### EXAMPLE: 3

This example demonstrate the use of an amplification product of the size close to that of primer dimer for nucleic acid target amplification result in higher amount of amplification product.

In order to demonstrate that amplification of an amplification product of the size close to that of primer dimer result in higher amount of amplification product, a 40 bp segment (base position 1114 to 1153) and 544 bp segment (base positions 560 to 1103) of gp 63 gene of Leishmania donovani were amplified in presence of [(□³² P] dATP as tracer using the amplification primers seq. id nos. 10 and 13 and seq.nos.14 and 15 respectively using 50 ng of chromosomal DNA and 60° C. annealing temp for 10, 15, 20, 25 and 30 cycles. The amplification products were separated by polyacrylamide gel electrophoresis and the gels were analysed in phosphor imager. The no. of [□³²P] dATP that could be incorporated into the 544 bp product is 30 times that in case of 40 bp product. There were higher amount (10-20 times) of amplification product formation in case of 40 bp product in comparison to 544 bp product (FIG. nos. 23 and 24 and FIG. nos. 26-31).

### EXAMPLE-4

This example demonstrates that the use of an amplification product of the size close to that of primer dimer for nucleic acid amplification based analysis can make the analysis faster and can result in higher throughput.

In order to demonstrate that use of an amplification product of the size close to that of primer dimer for nucleic acid amplification based analysis, can make the target analysis faster the 40 bp segment (base position 1114 to 1153) and 64 bp segment (base position 1090-1153) of gp 63 gene of Leishmania donovani were amplified with the help of the amplification primers seq. id Nos. 10 & 13 and seq. id Nos. 17 & 13 in presence of a [□³²P] dATP as tracer with shorter annealing time and without any extension step. A denaturation time of 10 seconds and annealing time of 2 seconds was sufficient for the amplification and no separate extension step was required; because of which cycling time reduced considerably. There was good amplification of specific amplification products. The products being of very small size denaturation temperature, also can be reduced further which again would result in shorter cycling time. It is possible annealing time for cycling can be reduced further. Because of shorter cycling time and need for no final extension step amplification of a product of the size close to that of primer dimer result in a faster or high throughput PCR analysis. Further use of very short annealing time eliminate the formation of primer dimer and nonspecific product formation. Even the primer pair sequence id nos.6 & 16 which form primer dimer did not form any primer dimer in this cycling condition (FIG. 25). it has also been observed that formation of primer dimer reduces with reduction of annealing time for the primer pair seq id nos. 6 & 16, annealing time of 5 seconds or less eliminated formation of primer dimer totally. Reduction of annealing time reduces the yield of PCR product. However, use of reduced annealing time for early 10-20 cycles and little longer annealing time for remaining cycles eliminates primer dimer formation without affecting yield of the PCR product. For this fluorescent primer sequence id no.19 and the primer sequence id no.13 were used with 50 nanogram template DNA in an amplification reaction with 10 seconds denaturation and 4 seconds annealing at 55° C. for 30 cycles and in another amplification reaction with 10 seconds denaturation and 4 seconds annealing at 55° C. for 20 cycles and 10 seconds denaturation and 15 seconds annealing for remaining 10 cycles. There was amplification in both cases and increase in yield of the amplification product in second case. All the amplification reactions were carried out at default ram rate of 0.1° C./second for cooling and 0.1° C./second for heating. It took only 50 minutes to complete a 30 cycles PCR amplification run, which is much less than the time taken for any standard 30 cycles PCR amplification run (normally 2-3 hours). Presently there are thermal cycler machines available with ram rates of 3-5° C./second for heating and 2-3° C./second for cooling. By using higher ram rate of heating and cooling a 30 cycles PCR amplification run can be completed in 15-30 minutes, which is again still faster. Further ordinary PCR tubes and 96 tubes trays can be used for the analysis thus making the amplification/analysis much faster increasing throughput. There is only one machine (Idaho make) where a 30 cycles amplification reaction can be completed in 20-30 minutes but the same machine require a special glass tubing for sample loading and only 20 samples can be analyzed at a time which are again inconvenient and reduces throughput.

### EXAMPLE: 5

This example demonstrate the use of an amplification product of the size close to that of primer dimer for nucleic acid amplification based analysis.

Amplification of an amplification product of the size close to that of primer dimer i.e. size of the forward primer plus size of the reverse primer plus zero to twenty five bases was chosen to evaluate whether amplification product of such size can serve the above purpose. For this amplification of products of such size from different regions of Leishmania donovani gp 63 gene was carried out. One of such segment, the 40 bp segment (base positions 1114-1153) was amplified using the forward primer sequence id no.10 and as reverse primer sequence id no 13 or sequence id nos. 11 or 12. This amplification was carried out hundreds of times with low and extremely high stringency of amplification with different concentrations of enzymes and DNA. Specific amplification product got amplified every time without a single failure and consistent in comparision to bigger size product. Non-specific amplification product was also not formed even with use of higher concentration of the enzyme. Higher quantity of DNA could be used without formation of any non-specific amplification product. Primer dimer formation was not observed with primer concentration less than 0.2 μm and annealing time of one minute. Reduction of annealing time will ensure further non-formation of primer dimer. It was also observed that employment of very short annealing time for first 10-20 cycles and little longer annealing time for remaining cycles could solve all the above mentioned problems without any loss in the yield of the amplification product. Fluorescent labeled oligonucleotides primers were also used in the same condition with the same result. Hence, it was found that amplification of amplification product of the size close to that of primer dimer can be used for reliable target analysis, which can be easily adapted in routine clinical diagnostic laboratories.

### EXAMPLE: 6

This example is directed to the design of amplification primers for the amplification of an amplification product of the size close to that of primer dimer:

For the purpose, Leishmania donovani gp63 gene (GC content 70%) was chosen as target. A few primer pairs were picked up, checked in oligo design software and tested for absence of primer dimer formation, and secondary structure or loop formation. These primer pairs were so chosen that those could be labeled through T base modification and at least one of them could also be used as hair-pin primer with good stem structure stability. These primers were chosen randomly and are not high specificity primers. In fact one of the primers, i.e. seq. id no.13 has a six nucleotide palindromic sequence close to it's 3′ end Two of those pairs (seq. id nos. 10 & 13, 17 & 18) turned out to be reasonably good candidates. The hair-pin primers were designed for hair-pin stem and loop structures with different lengths of stem and were similarly tested for absence of primer dimer formation. Primer dimer formation using forward primer seq.no.10 and hair-pin reverse primers (seq.id nos.11 & 12, forming 8 & 9 base pair stems respectively) were checked separately. There were either much less than one percent in case of seq. id nos. 10 & 11 pair or no primer dimer formation in case of seq. id no.10 & 12 in absence of any template DNA (FIGS. 27-31). Hence it is possible to design primer pair for amplification of an amplification product of the size close to that of primer dimer even from highly GC rich target sequence and can be used for target amplification without formation of primer dimer.

It is demonstrated that suitable primer pairs can be designed for the amplification of a target sequence for the amplification product of the size close to that of primer dimer, i.e. the size of forward primer plus the size of the reverse primer plus zero to twenty five bases, for monitoring of an amplification reaction of a target sequence, which can be monitored by sensitized emission (FRET) as well as by many other monitoring methods.

While designing primers that do not give primer dimer product one has to avoid base complementarity among the first three to six bases at 3′ end of the primers so that the two primers cannot sit on each other and get extended. The primers can be checked in many primer design software like Oligo-4, amplify 1.3, primer premier etc. Further we observed that primers having high GC content, stretches G and C near 341 ends formed primer dimer even though those did not have much base complementarity at their 3′ end, and GC contents of approximately 50 percent (between 45 and 55 percent) among the six to ten bases at the 3′ end of both the primers or at least one primer of the primer pair helps reducing to negligible level or eliminating primer dimer formation. All primers were checked for primer dimer formations at different temperatures in absence of any target sequence at primer concentration ranging from 0.18 to 0.4 μM, Taq DNA polymerase at conc.>3 units per 100 □l reaction volume whereas normally 2-2.5 U is routinely used and an ordinary micro pipette and ordinary tips were used leaving room for dispensing of excess enzyme this was done deliberately for checking to what extent dispensing errors affect the amplification of specific product, non-specific product and primer dimer formation. Use of less enzyme helps in reducing primer dimer formation. Primer dimer formation was negligible or absent for the primer pair oligo seq. id nos. 10 and 13, and 17 and 18 at primer concentration of approximately 0.2 μM.

Primers should be designed preferably from a region of approximately 50 percent (45-55) GC content, lest region encompassing 10 to 20 base region with approximately 50 percent GC content so that six to eight bases at 3′ ends of both the primers or at least one of the primers can have approximately 50 percent GC content avoiding more than two G or C or combination thereof. Higher Tm of primers should be preferred. Moreover internal fluorophore labeling near 3′ ends of two oligonucleotide primers help avoiding primer dimer formation. Primer pairs FAM labeled forward primer sequence id no.19 and JOE labeled reverse primer sequence id no.20 did not form any primer dimer in 0.4 micro molar concentration of the primers. The oligonucleotide primers for FRET based detection should be designed for labeling internally with fluorophores 2-4 nucleotides away from 3′ ends for elimination of primer dimer formation and at least 2 nucleotides away from 3′ ends for good extension of the primers. Fluorophore labeling at 3′ end inhibits amplification reaction as a result can lead to primer dimer formation and non-specific product formation.

In case of hair-pin primers, primers with different length of the stem 5 to 9 bases were checked for the stability of the structure in Zuker DNA folding analysis. Stem structures with higher thermodynamic stability were chosen and were checked for primer dimer formation. Primer dimer formation reduced with increasing length of the stem. Primers with relatively less stability of the stem structure (oligo seq. id no.11 of 8 base stem) resulted in very small amount of primer dimer formation, at primer concentration of 0.4 μM and almost negligible or no primer dimer formation at primer concentration of approximately 0.2 μM (in combination with oligo sequence id no.10) while primers with stable stem structure (oligo seq. id no.12 of 9 base stem) did not give any primer dimer formation at primer concentration of approximately 0.4 μM (in combination with the oligo sequence id no.10). May be sluggish or no opening of the stem of the hair-pin oligo nucleotide primers (oligo seq. id no.12) during annealing resulted in no primer dimer formation. However, both the hair-pin primers oligo seq. id nos. 11 and 12 resulted in specific amplification product formation in presence of the template DNA (FIG. nos. 26-31). Above amplification reactions were carried out in 1.5 mM MgCl₂ concentration, the concentration at which mostly PCR reactions are carried out. At this MgCl₂ conc. stem structure containing 8-9 base pairs may be stable enough. But at higher MgCl₂ conc. of 2.5 mM 5-6 base pair stems may be stable enough to reduce primer dimer formation. But higher MgCl₂ conc. can result in more non. Specific product formation in case of certain target sequence. But non-specific product formation would not affect target detection in case of sensitized emission based detection of this invention.(because of distance relationship between donor and acceptor moiety).

### EXAMPLE: 7

This example demonstrate the use of both oligonucleotide primers of amplification reaction as labeled oligonucleotide primers does not affect PCR amplification reaction much and internal fluorophore labeling near 3′ ends of both the forward and reverse primer avoid primer dimmer formation.

FAM labeled forward primers (seq. id no.19) and JOE labeled reverse primer (seq id no.20) as well as same forward and reverse primers as unlabeled oligonucleotide primer (seq. id no.10 & 13) were used in amplification at 0.35 μM (micro molar) concentration for labeled primers and 0.2 μM for unlabeled primers and an annealing temperature of 55° C. The sizes of the amplification products were same and the yield of the amplification product of labeled primers was slightly less in comparison to the yield of the amplification product of unlabeled primers (FIG. Nos.32 & 33).

FAM labeled forward primer (seq ID No.21) and JOE labeled reverse primer (seq ID No.20) as well as same forward and reverse primers as unlabeled oligonucleotide primers (seq.id nos. 10 & 13) were used in amplification reactions. The sizes of the amplification products were same and the yield of the amplification product of labeled primers was slightly less in comparison to the yield of the amplification product of unlabeled primers (seq.id nos.10 and 13). However, there was a primer dimer formation in the case of seq. id nos.20 & 21 and no primer dimer formation in the previous case (seq. id nos. 19 and 20) when used in absence of template. This primer dimer may be the homodimer of the FAM labeled forward primer (seq. id no.21) due to the inhibition of the amplification reaction for the 3′ end terminal FAM labeling of the primer (seq. id no.21). This demonstrate that oligonucleotide primer labeled internally at the extreme 3′ end can be extended by Taq DNA polymerase. This also demonstrate the internal fluorophore labeling near 3′ ends of both forward and reverse prime ends can be extended by Taq DNA polymerase and incorporated into the amplification product more or less with the sane efficiency as that with unlabeled. This also demonstrates that internal fluorophore labeling near 3′ ends of both the forward and reverse primers help in eliminating primer dimer formation.

In all three cases the yield of the amplification product utilizing fluorophore labeled primers was compared with the yield of the same amplification product using unlabeled primers. The yield of amplification product of the labeled primer appears to be a few fold less in comparison to that of the amplification product of unlabeled primers. In this case of comparison the unlabeled primer pair (seq. ID No.10 and seq. ID No.13 was also used at 55′ C annealing temperature along with the fluorophore labeled primers. Hence yield of the amplification product of unlabeled primers is expected to be higher than the same when 60′ C annealing temperature is used. In an amplification reaction annealing temperature used for fluorophore labeled primers should be atleast 5° C. less than that used for unlabeled primers. Hence actual yield difference would be much less than what has been observed. Use of small amount of unlabeled primers also will improve the efficiency of incorporation of the two labeled amplification primers into the amplification product and the yield of the amplification product. Further efficiency of utilization of flurophore labeled oligo nucleotide by DNA polymerase may depend on the linker length between the nucleotide and the fluorophore (Nucl. Acid Research 1994, 22 (16), 3418-3422). The yield of the amplification product of fluorophore labeled primers can be further improved by varying length of the linker linking the fluorophore to the oligonucleotide oligonucleotides. Oligonucleotide primers labeled internally with Fluorophores more-than two bases away from their 3′ ends were utilized in PCR reaction as efficiently as unlabeled oligonucleotide primers. Use of Rhod labeled oligonucleotide primer sequence id no.26 in association with the primer seq id no.19 gave good yield of amplification product.

### EXAMPLE: 8

This example is directed to the detection of an amplification product by fluorescence energy transfer (FRET) between donor flurophore FAM and acceptor flurophore JOE on two amplification primers of an amplification reaction.

To detect amplification product FAM labeled forward primer (Seq. ID No. 19) and JOE labeled reverse primer (Seq. ID No.20) were used in PCR amplification reaction with and without template DNA. After amplification reaction, amplification reaction mixtures were illuminated with FAM specific excitation light of 488 nm and characteristic emissions at 550 nm of JOE as well as emission of FAM 520 nm were measured. There was sizeable increase in JOE emission and decrease in FAM emission from the reaction mixture containing template DNA, whereas there was almost negligible increase in JOE emission from the reaction mixture containing no template DNA (FIG. 34). Use of primer seq. id no.26 in association with primer seq. id no.19 gave similar result.

### EXAMPLE: 9

This example is directed to the use of hair-pin quenched oligonucleotide reverse primer labeled with an acceptor fluorophore FAM near 3′ end and a quencher DABCYL at 5′ end and a donor fluorophore FAM labeled forward primer.

Amplification reaction was carried out with a donor fluorophore FAM labeled forward primer labeled internally near 3′ end (seq id no 19) and a hair-pin reverse primer labeled internally with an acceptor fluorophore FAM near 3′ end and a quencher DABCYL at 5′ end (seq id no 23) with and without template DNA (Leishmania donovani chromosomal DNA). After the amplification reaction the reaction mixtures were illuminated with FAM specific excitation light of 488 nm wavelength and characteristic emissions of FAM at 530 nm was measured.

There was large increase in FAM emission from the reaction mixture containing the template DNA whereas there was almost no increase in FAM emission from the reaction mixture containing no template DNA (FIG. 35) A signal to noise ratio of approximately 60 was observed. The Signal to noise ratio observed is much less then expected which may be due to breakage of the linkage between DABCYL and the oligonucleotide. Signal to noise ratio can be improved further by, improving the labeling and purification of the labeled primers particularly the fluorophore and quencher labeled primer. Labeling of the dual label primer seq. id no.23 with quencher DABCYL at 5′ end can be accomplished better through incorporation of DABCYL dT phosphoramidite during synthesis. Use of a suitable spacer for attaching the fluorophore to the oligonucleotide may result in still higher signal to noise ratio. Further the interstrand FRET energy transfer may be sequence dependent.

### EXAMPLE: 10

This example is directed to demonstrate the reduction of noise from primer dimer formation in FRET based detection or quantitation of amplification product or reaction:—

For reduction of noise from primer dimer the reverse hair-pin primer (seq. id no.23) was labeled near its 3′ end with a donor fluorophore FAM and at 5′ end with quencher DABCYL. The forward primer (seq. id no.24) was labeled near its 3′ end with the quencher DABCYL. The forward primer and the reverse primers were designed to amplify a 64 bp segment of Leishmania donovani gp 63 gene and the two primers were so designed that when incorporated into the amplification product the FAM of the reverse primer and the quencher DABCYL on both forward as well as reverse primer remained more than 15 base away from FAM on either side thus allowing FAM incorporated into the amplification product to emit its own characteristic emission, which could be measured. In case of primer dimer formation the quencher DABCYL near 3′ of the forward primer would come proximal (within FRET distance) to the fluorophore FAM resulting in quenching of FAM emission, thus reducing or nullifying contribution of primer dimer if any towards fluroescence emission from FAM due to separation of FAM and DABCYL of forward primer. A signal to noise ratio of 50 was observed (FIG. 36). Signal to noise ratio was much less than expected higher value due to presence of free FAM labeled primer in the FAM-DABCYL double labeled primer. Signal to noise ratio can be improved further by improving the primer labeling and purification method.

### EXAMPLE: 11

This example is related to a Close tube format detection

In this Leishmania donovani chromosomal segment was amplified for 10,15,20,25 & 30 cycles using FAM labeled forward primer (Seq. ID No. 19) and FAM. and quencher DABCYL labeled hair-pin quenched reverse primer (Seq. ID No.23) in presence of 100 ng. of Leishmania donovani chromosomal DNA, primers at a conc. of 3 5OpM. As a control same amplification reaction was carried out in absence of template DNA. The primers were designed such that fluorescence resonance energy transfer signal is generated only when the amplification product is formed, i.e. the forward and reverse primers get incorporated into the two opposite strands of the amplification product in right proximity. There was increase in signal to noise ratio after 15 cycles and further increase there after. This allows real time quantification of target nucleic acid sequence. This method eliminates the carry-over contamination problem associated with PCR, simplifies the process.

### EXAMPLE: 12

This example is directed to demonstrate that an inter strand sensitized emission can be used for monitoring of PCR amplification reaction using labeled primer and labeled probe

To demonstrate that sensitized emission could be used to monitor PCR amplification reaction or amplification product, the synthetic template (Sequence given in FIG. 8) was amplified using the FAM labeled forward primer (Sequence id no.5) and the reverse primer (Sequence id no 2) in the presence of 3′ JOE labeled probes which bring the JOE label at distances of 5, 10, 15, 20 bases from FAM label in amplified product. After PCR amplification the tube was denatured and annealed once more and the amplified product was measured by illuminating the reaction mixture with FAM excitation wavelength of 488 nm light and measuring the emission of JOE at 553 nm at 37-40° C. There was a decrease in FAM emission (i.e., quenching of donor fluorescence) and increase in JOE emission. Energy transfer was observed upto the distance of 20 base pair with gradual decrease and maximum energy transfer was observed at a distance of 5 base pair. The JOE labeled probes are not shown.

### EXAMPLE: 13

This example is directed to a Heterogeneous phase target detection using PCR amplification:—

For heterogeneous phase PCR amplification reaction first amplification primer is fixed through a linker and a spacer to the surface of glass, the solid phase and the second amplification primer remains in solution along with all other components. The efficiency of PCR will depend on the linker and spacer length and the tethering density of fixed oligonucleotide primer. Many methods are known in the art for attaching oligonucleotides to solid surface. 5′ end phosphorylated oligonucleotide primer was attached to small glass chips surface modified with aminopropyl silane using spacers of different lengths and diN-hydroxy succinamide derivative of suberic acid as linker. Spacers used were hexamethylene diamine, a eighteen carbon diamino (at two terminal ends) spacer made from succinic acid, hexamethylene diamine and ethylene diamine and a polythene glycol (50 monomers) based spacer with amino groups at two ends. Spacers were attached to 5′ end phosphorylated oligonucleotide primer by standard procedures known in the art. The oligonucleotide primer with attached spacer was used at concs. of 5-10 μm for attaching to glass surface. Density of primer attached to glass surface was measured by using ³²P end labeled primer and extent of amplification was measured by using usual 200 μm conc. of all four nucleotides and a trace amount of α-³²P-d ATP and measuring the amount of 32P d ATP incorporated into the amplification product that remained attached to-glass chip and was later removed by restriction digestion. Control reaction was carried out without template. There was increase in the efficiency of the amplification reaction with the increase in the spacer length. Very little amplification took place with the use of hexamethylenediamine spacer. Though there is a report of oligonucleotide attachment density of 100 femtomole/sq mm using phenylene di-isothiocyanate as linker we could get only upto 10 femtomole/sq mm of primer density on glass chip. We could get upto 0.1% incorporation of α-³²P-d ATP into the amplification product of size approximately 800 bp from E coli chromosome using oligonucleotide primers seq. id no.27 and seq. id no.28, seq. id no.28 being used as tethered primer. There was no amplification reaction when amino modified paper was used as solid support. Considering 10 micron distance from the surface as reaction zone, the reaction volume per sq mm area will be 0.1 nano litre and the conc. of the primer attached to the solid surface will be 100 μm. Reactions were carried out in 50 μl volume. 0.1% incorporation of nucleotides into amplification product amount to 12 ng of DNA synthesis, which could be easily detected by using fluorescence/luminescence as a measurement mode. Use of amplification of a product close to the size of that of primer dimer would further improve the efficiency of the amplification reaction. Hence solid surface based PCR amplification can be used for target quantitaion. Advantage of this hetrogeneous phase PCR is that a large number of targets can be analysed simultaneously in in-situ PCR format thus reducing the cost drastically, particularly in case of luminescence based detection It can be used for real time monitoring or quantitaion of nucleic acid amplification targets. This can be also be useful for large scale or high through put real time quantitation of expressed RNA sequences for quantitative RNA expression profiling. For high through put real time RNA expression profilling a universal primer common for all mRNAs in the sample that remain in solution phase should be used in combination with specific primers for individual mRNAs designed from sequences at 5′ end of the mRNAs or a segment of the cDNAs generated by restriction digestion. The common universal primer is designed from a common additional sequence ligated to the 5′ end of the cDNAs or a restriction fragment of the cDNAs. Many other variations are possible. Monitoring of the amplification can be carried out by using suitable fluorophore labeled primers for FRET based and other detection. Right now there is no instrument for the same.

### EXAMPLE: 14

Under this Example the selective advantages in amplification close to primer dimer in terms of higher yield and specificity is demonstrated:

For the purpose oligonucleotide primers were designed from E. coli genome sequence for amplification of a 50 base pair size and a 504 base pair size segments of E. coli genome. The primers were designed for the above amplification with Sequence ID Nos 29 to 31.

Amplification reactions were carried out by employing 30 cycles of one minute denaturation at 94° C., one minute annealing at 54°C., and one minute extension at 72° C. and 7 minute final extension at 72° C. The primers were used at a conc. of 0.2 μM.

Result

In absence of template DNA there was formation of no primer dimer in the amplification condition used Amplification of 50 base pair size product (close to primer dimer size) did not give any non-specific product while the 504 base pair size product gave non-specific product. The yield of the 50 base pair size product was at least 6-8 times that of the 504 base pair size product under the same

### DESCRIPTION OF THE FIGURES

FIGS. 1A and 1B The structure of the hair-pin oligonucleotides of the invention in closed quenched (a) and open signal emitting (b) states; open circle (F) being the donor or acceptor fluorophore and the solid-circle being the quencher.

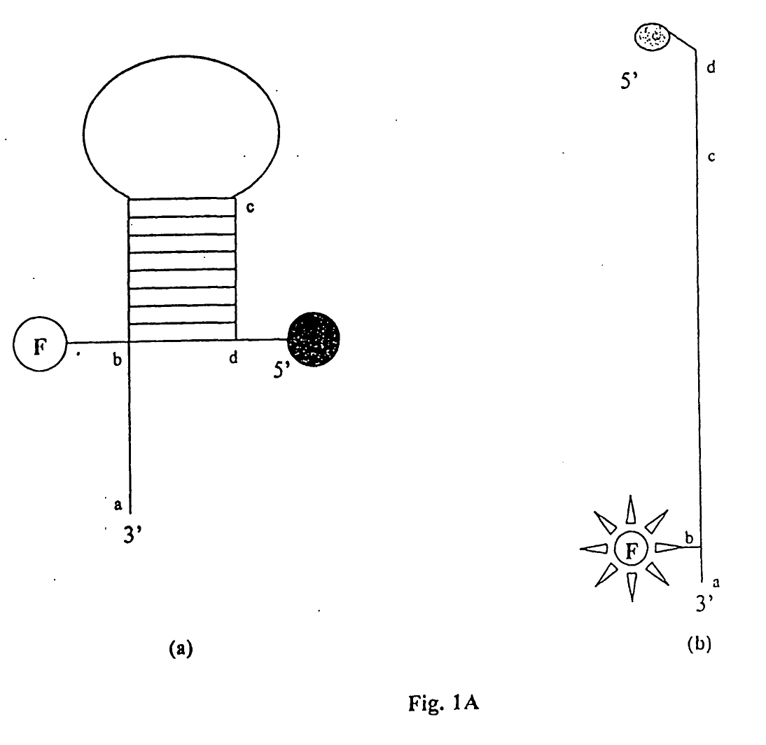

FIG. 2 Schematic illustration of the use of donor fluorophore labeled linear forward primer and acceptor labeled linear reverse primer in the detection and/or quantitation of an amplification product produced from PCR amplification. A fluorescence energy transfer signal is generated only when the fluorophore labeled primers get incorporated into the two strands of the double stranded amplification product, (D) donor fluorophore (A) acceptor fluorophore.

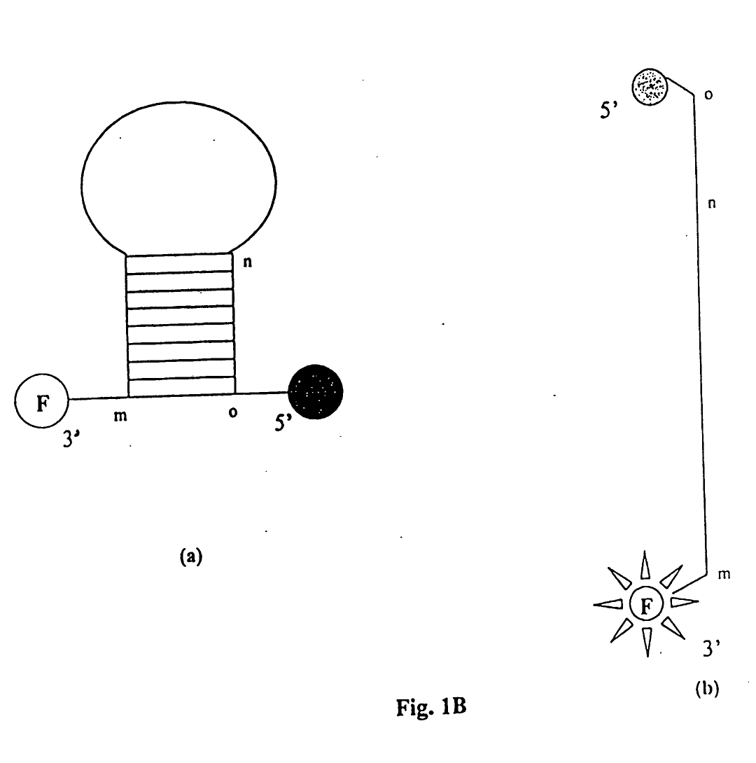

FIG. 3 Schematic illustration of the use of donor and acceptor labeled quenched hair-pin primers in PCR amplification, (A) is acceptor, (D) is donor, (Q) is quencher.

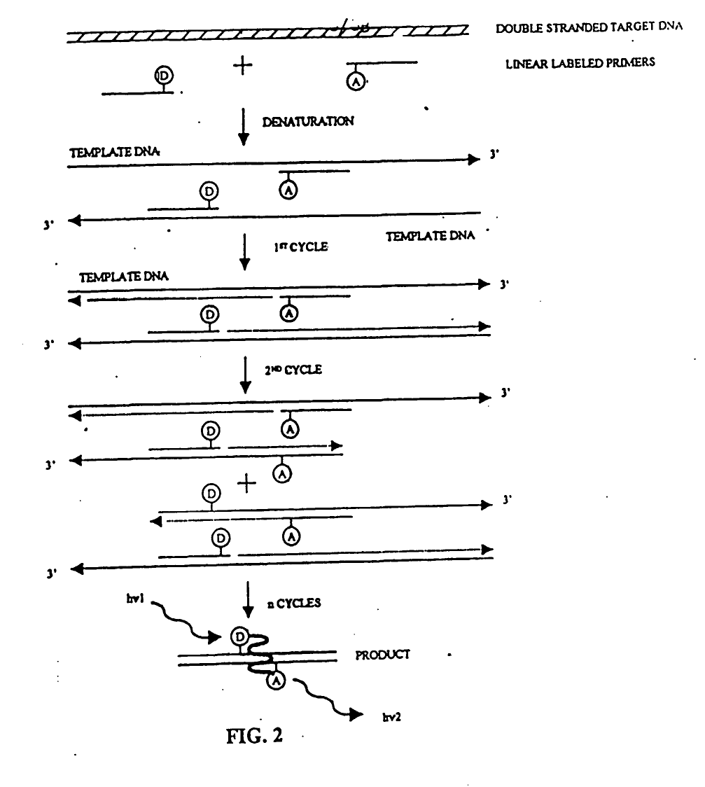

FIG. 4 Schematic illustration of linear donor labeled forward primer and acceptor labeled quenched hair-pin primer in PCR amplification (A) acceptor fluorophore, (D) is donor, (Q) is quencher.

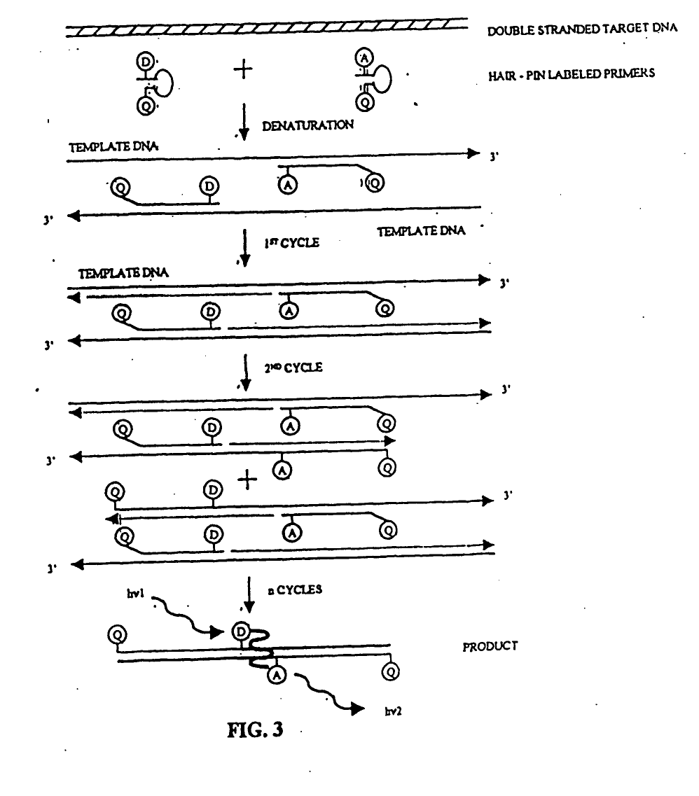

FIG. 5 Schematic illustration of the use of unlabeled reverse primer, donor labeled quenched hair-pin forward primer and acceptor labeled quenched hair-pin probe in PCR amplification.

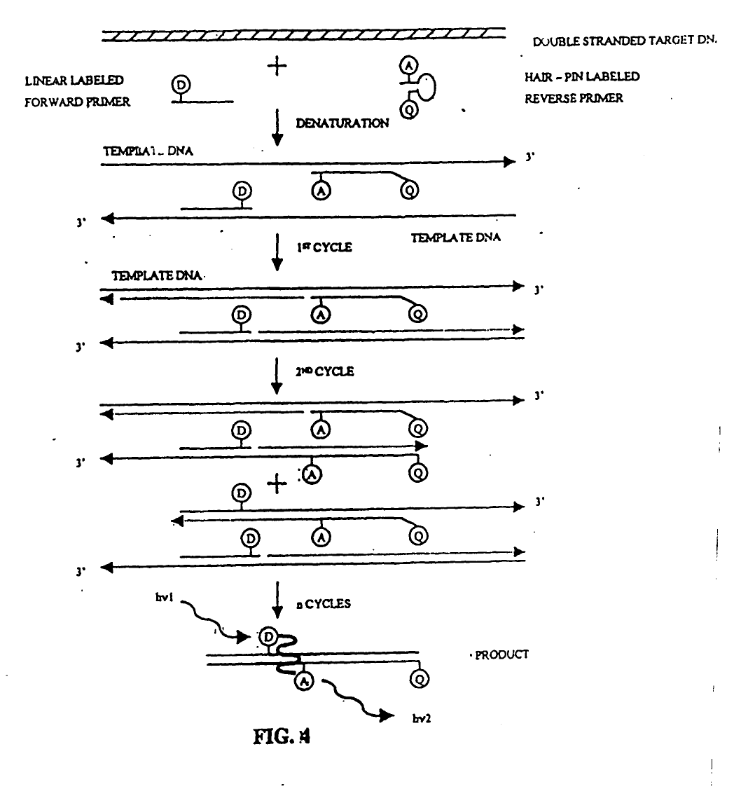

FIG. 6 Schematic illustration of the use of donor fluorophore labeled linear forward primer, acceptor fluorophore labeled hair-pin quenched reverse primer and blocker in triamplification. A fluorescence resonance energy transfer signal is generated only when the donor fluorophore labeled forward primer and acceptor fluorophore labeled reverse primer gets incorporated into the two strands of the amplified product; (D) donor fluorophore (A) acceptor fluorophore and (Q) quencher.

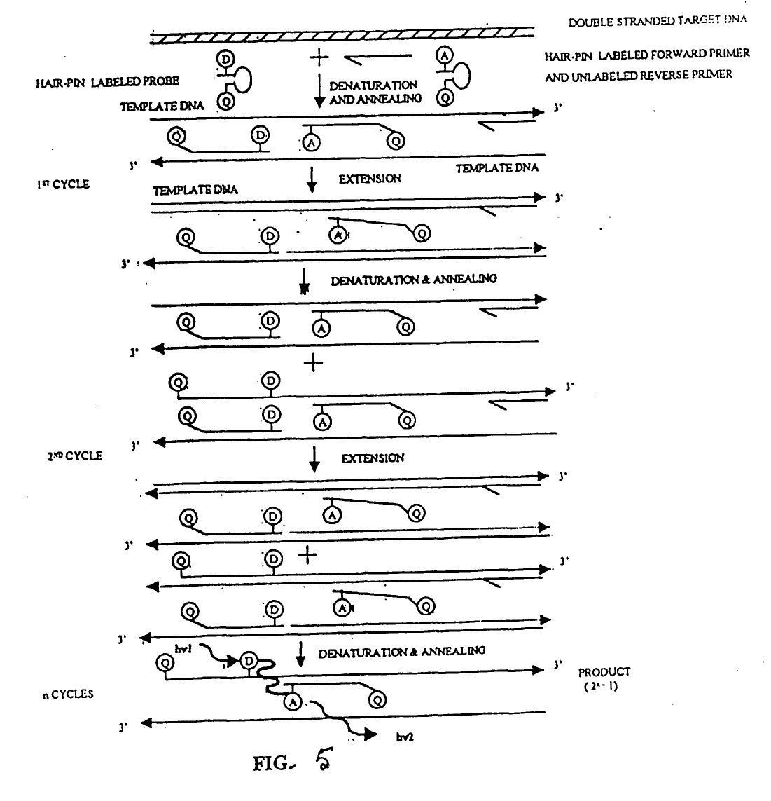

FIG. 7 Schematic illustration of the use of acceptor fluorophore labeled hair-pin quenched forward and donor fluorophore labeled reverse primer in nucleic acids sequence based amplification (NASBA). A fluorescence resonance energy signal is generated only when the acceptor labeled forward primer and donor labeled reverse primer get incorporated into the two strands of the amplification product (D) donor (A) acceptor (Q) quencher.

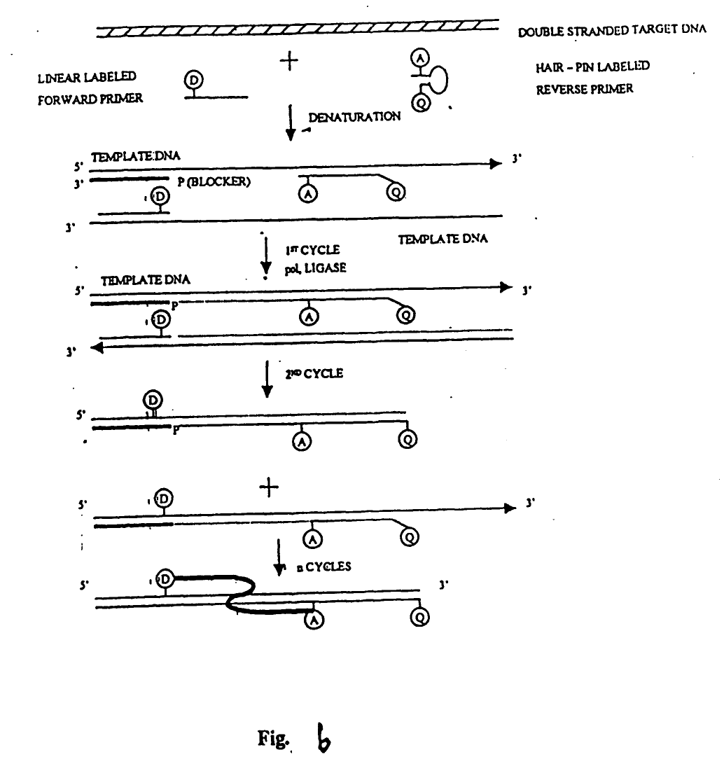

FIG. 8 Sequence of 70 bp synthetic template DNA

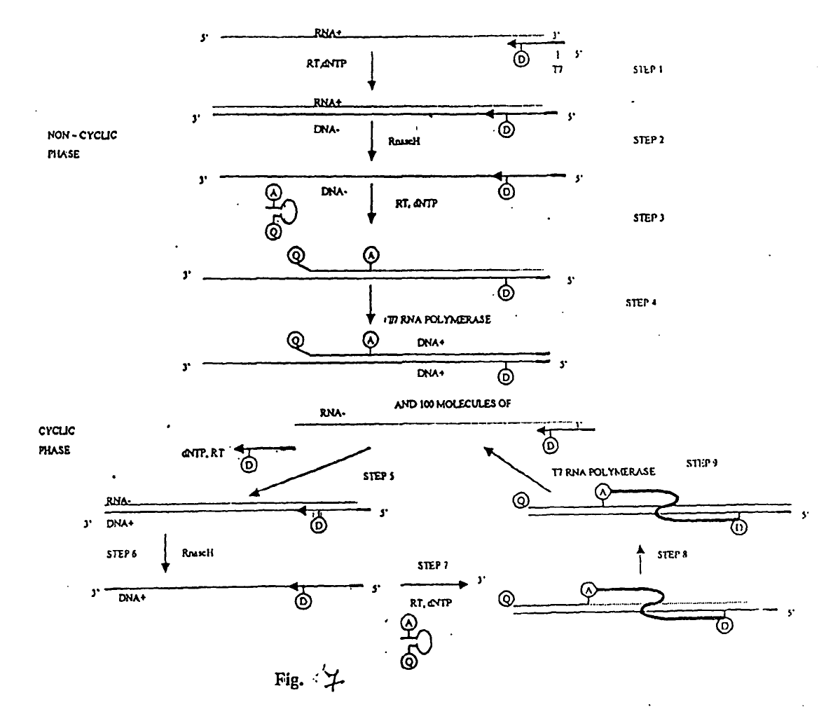

FIG. 9 Sequence of the 40 bp segment (base position 1114-1153) of the Leishmania donovani gp 63 gene (Gene Accession No.M60048).

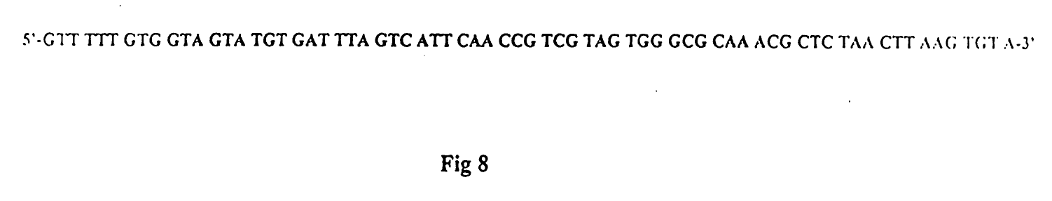

FIG. 10 Sequence of 40 base pair segment (base position 566-605) of the Leishmania donovani gp 63 gene (Gene Accession No.M60048)

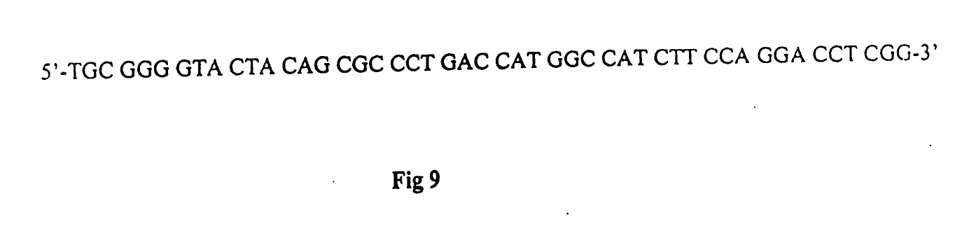

FIG. 11 sequence of 36 base pair segment (base position 1094-1129) of the Leishmania donovani gp63 gene(Gene Accession No.M60048)

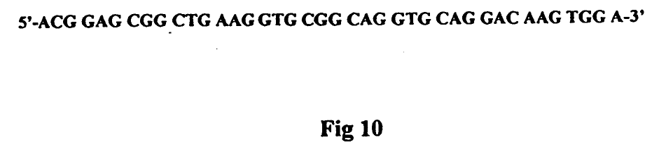

FIG. 12 FAM labeled oligonucleotide primer for amplification of 70 bp synthetic template.

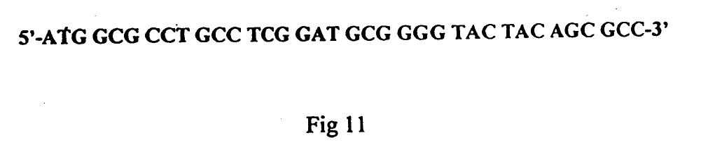

FIG. 13 FAM labeled forward primer (seq.no.19) for amplification of 40 bp segment (base position 1114-1153) of Leishmania donovani gp63 gene.

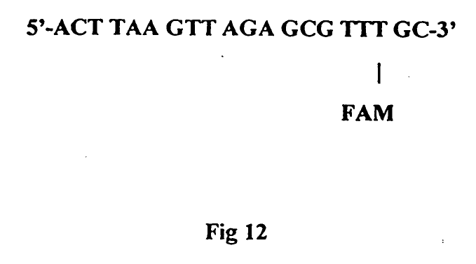

FIG. 14. JOE labeled reverse primer (seq.no.20) for amplification of 40 bp segment (base position 1114-1153) of Leishmania donovani gp63 gene.

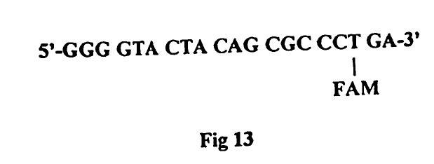

FIG. 15. FAM labeled forward primer (seq.no.21) for amplification of 40 bp segment (base position 1114-1153) of gp63 gene of Leishmania donovani.

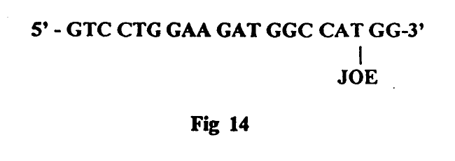

FIG. 16 JOE and DABCYL labeled reverse primer seq.no.22 for amplification of 40 bp segment (base position 1114-1153) of Leishmania donovani gp63 gene.

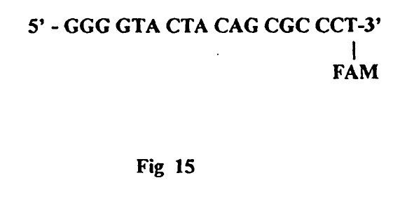

FIG. 17 FAM and DABCYL labeled reverse primer (seq.no.23)

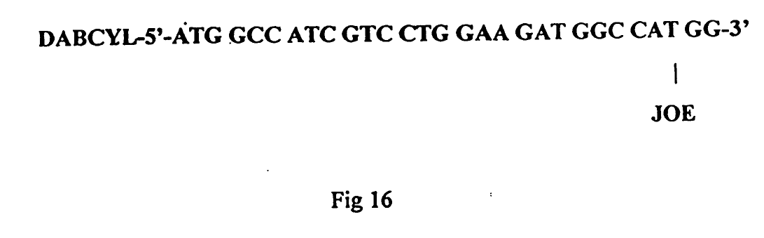

FIG. 18 DABCYL labeled forward primer (seq.no.24)

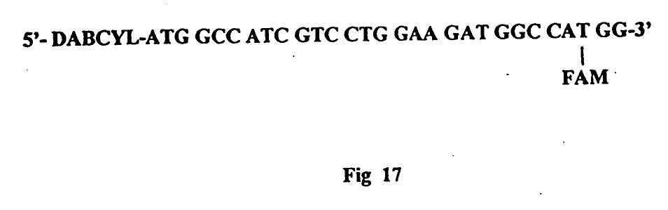

FIG. 19 sequence of 610 base pair segment (base position 560-1170) of the Leishmani donovani gp63 (Gene Accession No.M60048)

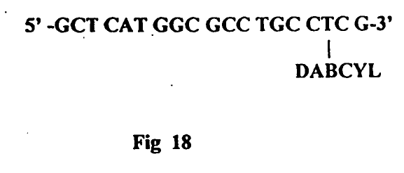

FIG. 20 Gel image illustrates that amplification of specific amplification product and no primer dimer is formed using primer pairs seq.nos.8 and 13; 14 and 15 and 10 and 13. From left to right lane no.1 and 2 amplification product (66 base pair) of primer pair seq.nos.8 and 13 in duplicate, lane no.3 and 4 amplification product (544 bp) of primer pair seq.nos.14 and 15 in duplicate, and lane no.5 and 6 amplification product (40 bp) of primer pair seq.nos. 10 and 13 in duplicate.

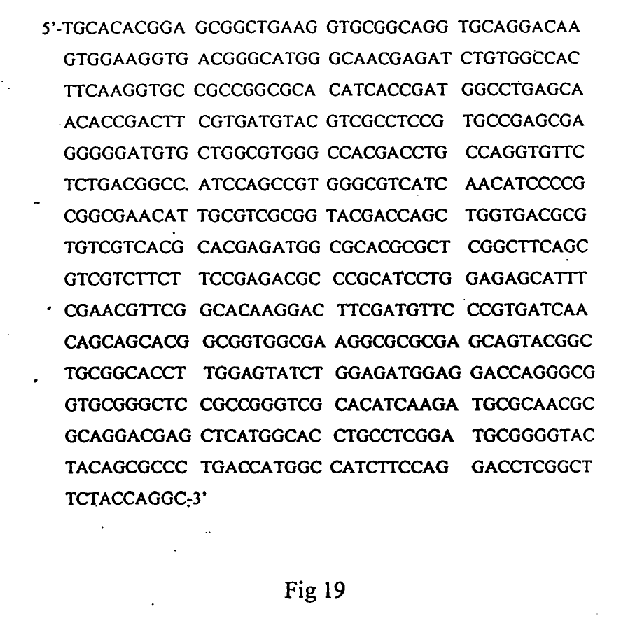

FIG. 21 Gel image illustrates use of an amplification product of the size close to that of primer dimer for nucleic acid amplification helps in eliminating or reducing non-specific amplification product formation. Upper gel image is image of the gel at lower sensitivity while the lower gel image is the same image at higher sensitivity of the phosphor imager. From left to right lane no. 1 and 2 amplification product of the primer pair seq.no.6 and 7 in duplicate, lane no.3 and 4 amplification product of the primer pair seq.no.8 and 9 in duplicate lane no.5 and 6 amplification product of the primer pair seq.no.10 and 13 in duplicate, lane no.7 and 8 amplification product of the primer pair seq.no.8 and 13 in duplicate, lane no.9 and 10 amplification product of the primer pair seq.no.14 and 15 in duplicate.

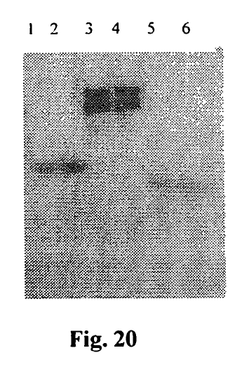

FIG. 21A—Gel image also illustrates use of an amplification product of the size close to that of primer dimer for nucleic acid amplification helps in eliminating or reducing non-specific amplification product formation. From left to right lane no. amplification product of the primer pair seq.no.6 & 16 in absence of the template DNA, lane no.2 amplification product of the primer pair seq.no.6 & 16 in presence of the template DNA, lane no.3 and 4 amplification products of the primer pair seq.no.13 and 17 in absence and in presence of template DNA respectively, lane no.5 amplification product of the primer pair seq.no.10 and 13 in absence of template DNA, lane no.6 and 7 amplification product of the primer pair seq.no.10 and 13 in presence of template DNA in duplicate, lane no.8 and 9 amplification product of the primer pair seq.no.17 and 18 in absence and in presence of template DNA respectively.

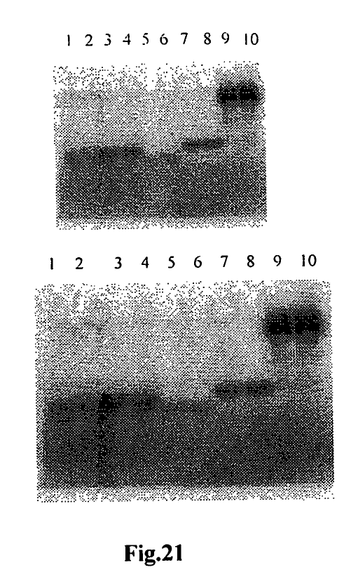

FIG. 22 gel image illustrates use of an amplification product of the size close to that of primer dimer for nucleic acid amplification helps in eliminating or reducing non-specific amplification product formation. Upper gel image is image of the gel at lower sensitivity while the lower gel image is the same image at higher sensitivity of the phosphor imager. From left to right lane no.1 and 2 amplification product of the primer pair seq.no.14 and 15 in duplicate, lane no.3 and 4 amplification product of the primer pair seq.no.6 and 15 in duplicate, lane no.5 and 6 amplification product of the primer pair seq.no.10 and 13 in duplicate, lane no.7 and 8 amplification product of the primer pair seq.no.14 and 9 in duplicate, lane no.9 and 10 amplification product of the primer pair seq.no.14 and 13 in duplicate.

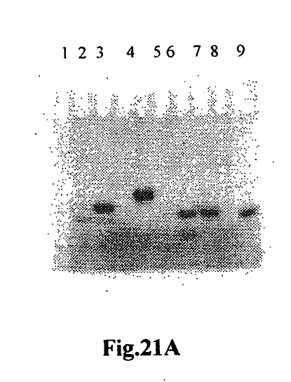

FIG. 23 Gel image illustrates use of amplification product of the size close to that of primer dimer for nucleic acid target amplification result in higher amount of amplification product. The amplification product of primer pair 14 and 15 in presence of 50 ng of L. donovani DNA was analyzed by 10 percent PAGE. Gel image from left to right lane no.1 and 2 amplification product formation after 10 cycles, lane no.3 and 4 same after 15 cycles, lane no.5 and 6 same after 20 cycles, lane no.7 and 8 same after 25 cycles, lane no.9 and 10 same after 30 cycles.

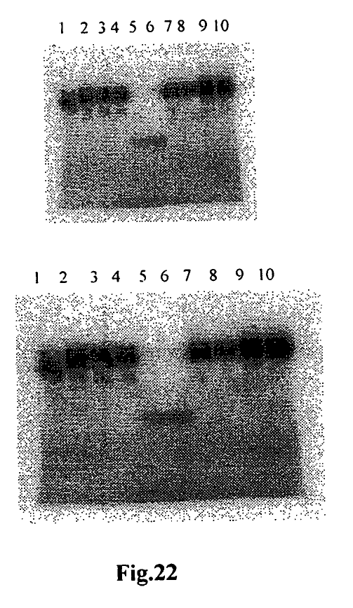

The product formed in the beginning, i.e., in the early cycles is non-specific product of size close to 100 base pair. The specific product, a high molecular weight product, is formed in later cycles and is the top most but one band.

FIG. 24 Gel image illustrates use of amplification product of the size close to that of primer dimer for nucleic acid target amplification result in higher amount of amplification product. The amplification product of primer pair 10 and 13 in presence of 50 ng of L. donovani DNA Gel image was analyzed 15 percent PAGE. From left to right lane no.1 and 2 amplification product formation after 10 cycles, lane no.3 and 4 same after 15 cycles, lane no.5 and 6 same after 20 cycles, lane no.7 and 8 same after 25 cycles, lane no.9 and 10 same after 30 cycles.

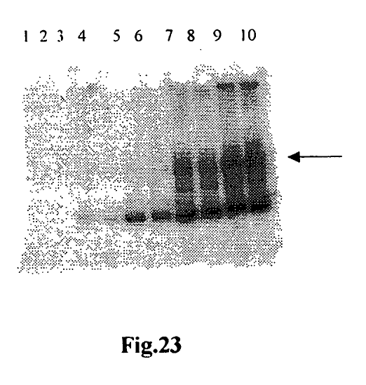

FIG. 25 Gel image use of an amplification product of the size close to that of primer dimer for nucleic acid amplification based analysis can result in higher throughput. From left to right lane-1, DNA marker for primer dimer formation between the primers seq.6 and seq.no.16. Lane-2, primer dimer formation by the primers seq.no.6 and seq. 16 in absence of template DNA lane-3, amplification product formation by the primer seq.no.6 and seq.no.16 in presence of Leishmania donovani chromosomal DNA (100 ng). Lane-4, primer dimer formation between the primers seq.no.17 and seq.no.13 in absence of template DNA Lane-5, amplification product formation by the primers seq.no.17 and seq.13 in presence of Leishmania donovani DNA. Lane-6, primer dimer formation between the primers seq.no.10 and seq.no.13 in the absence of template DNA. Lane-7, amplification product formation by the primers seq.nos.10 and 13 in presence of L. donovani DNA. Lanes 9 & 10 primer dimer formation and 100 bp amplification product formation by primers designed from [symbol] DNA. A denaturation time of 10 secs and temp 95° C. and annealing time of 2 secs and annealing temperature of 60° C. were employed for 30 cycles and was analyzed on 15 percent PAGE.

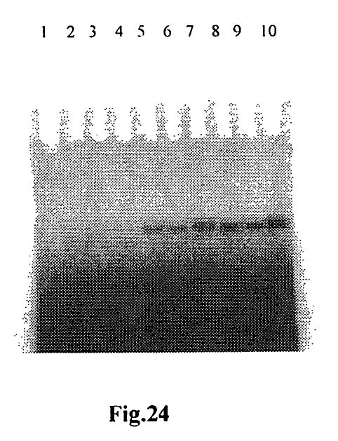

FIG. 26. The gel (20 percent PAGE) well no.1 primer seq.nos. 14 and 15, 0.35 μM each, L.donavani chromosomal template DNA 100 ng. Well No.3 44 bp DNA marker, well No.5 and 6 primer seq.nos.10 and 11, 0.1 8 μM each and L. donavani chromosomal template DNA 100 ng. Well no.8 and 9 primer seq.no.10 and 11 0.18 μM each and no L. donovani chromosomal template DNA Lower bands are small non-template dependant primer extension products or/extension of the primer seq.id no.11 over the primer seq. id no.11 near 5′ sequence forming the stem structure.

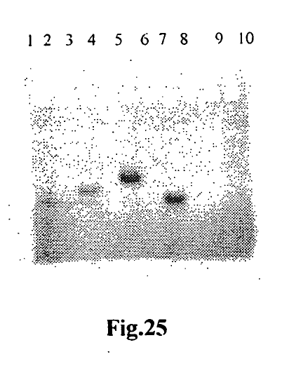

Doublet products may either be two amplification products one being single A moieties added at 3′ ends or most probably due to formation of smaller product from the designed amplification product by the primer seq. no.11. Primer seq no.11 inspite of having six nucleotide complementation did not form primer dimer in absence of template DNA.

FIG. 27. The gel (20 percent PAGE) well no.2 primer seq.nos.14 and 15 0.35 μM each, L. donovani chromosomal template DNA 100 ng. Well no.4 and 5 primer seq.nos.10 and 13, 0.18 μM each and L. donovani template DNA 100 ng. Well no.7 and 8 primer seq.no.10 & 13, 0.18 μM each and no template DNA well 10, 44 bp DNA marker.

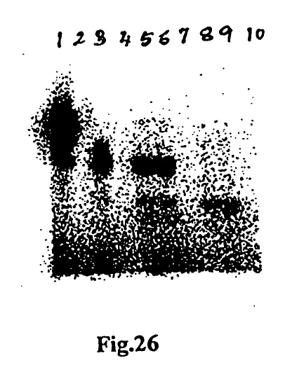

FIG. 28. Left gel (20 percent PAGE) well no.1 primer seq.nos.14 and 15, 0.35 μM each and L. donovani DNA 100 ng. Well 3 and 4 primer seq.nos. 10 and 12, 0.18 μM each and L. donovani DNA well 6 and 7 primer seq.nos.10 and 12, 0.18 μM each and no template DNA well no.9-44 bp DNA marker. Right gel (20 percent PAGE) well no.1—primer seq nos.14 and 15, 0.35 μM each and L. donovani template DNA 100 ng. Well nos.3 and 4 primer seq.nos. 10 and 12, 0.35 μM each arid L. donovani template DNA 100 ng. Well nos. 6 and 7—primer seq.nos. 10 and 12, 0.35 μM each and no template DNA. Well no.9-44 bp DNA marker.

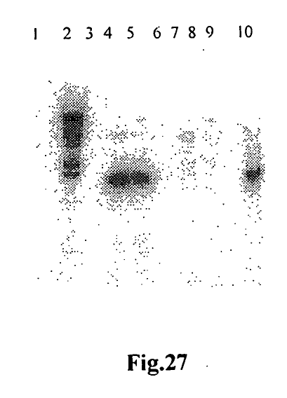

Doublet products may either be two amplification products one being single A moieties added at 3′ ends or most probably due to formation of smaller product from the designed amplification product by the primer seq. no.12. Primer seq no.12 inspite of having six nucleotide complementation did not form primer dimer in absence of template DNA.

FIG. 29 Upper left gel well no.1 and 2 primer seq.nos. 10 and 12, 0.35 μM each and 100 ng. L. donovani chromosomal DNA. Well no 4 and 5 primer seq nos.10 and 11, 0.18 μM each and 100 ng L.chromosomal DNA. Well 7 and 8 primer seq nos.10 and 11, 0.35 μM each and 100 ng L.chromosomal DNA. Well 10-44 bp DNA marker (20 percent PAGE). Upper right gel well no.1 and 2-primer seq nos 10 and 13 0.4 μM each and no template DNA. Well nos.4 and 5—primer seq nos.10 and 12, 0.35 μM each and no template DNA. Well nos. 7 and 8—primer seq nos.10 and 11, 0.18 μM each and no template DNA. Well no.10—44 bp DNA marker (20 percent PAGE). Lower gel well 1 and 2—primer seq nos 14 and 15, 0.35 μM and 100 ng L. donovani DNA. Well 4 and 5—primer seq nos 10 and 13, 0.35 μM each and 100 ng L. donovani DNA. Well no.7—44 bp DNA marker (20 percent gel). In the gels of the FIG. 29, ³²P labeled dATP has not got out of the gel.

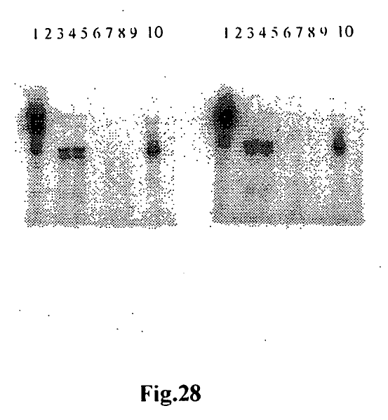

FIG. 30. 15 percent denaturing PAGE well no 2 and 3, 5′ end labeled primer seq no.11 and cold primer seq.no.10, 0.18 μM each plus L. donovani chromosomal template DNA. Well no.5-5′end label primer seq no.11 well no.6-5 end labeled primer seq no.,10 well no 7-44 bp marker DNA well no.9 and 10-5′ end labeled primer seq.no.10 and cold primer seq no.11 each 0. 18 μM plus L. donovani chromosomal template DNA. Well no.12 & 13-5′ end labeled primer seq.no.11 and cold primer seq.no.10 each 0.18 μM and no template DNA. Well no.15 —44 bp DNA MARKER. Well no 17 and 18-5′ end labeled primer seq 10 and cold primer seq no.11 each 0.18 μM and no template DNA.

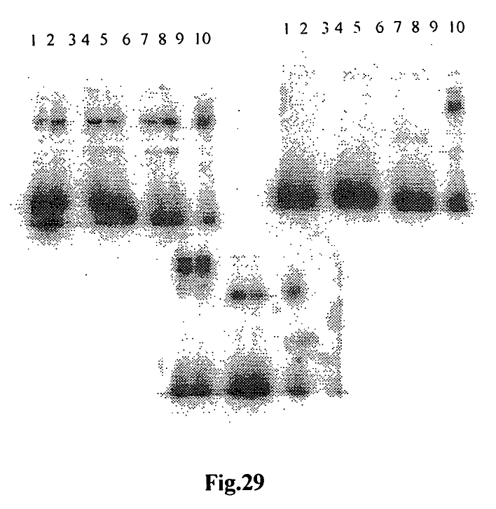

FIG. 31 15 percent denaturing PAGE well No.1 and 2-5′ end labeled primer seq no.11 and unlabeled primer seq no.10 each 0.18 μM without template DNA. Well no.4-44 bp DNA marker. Well no.5-5′ end labeled primer seq no.10. Well no.6-5′ end labeled primer seq no.11. Well no.8 and 9-5′ end labeled primer seq.no.11 and unlabeled primer seq.no.10 each 0.18 μM and L. donovani template DNA 100 ng.

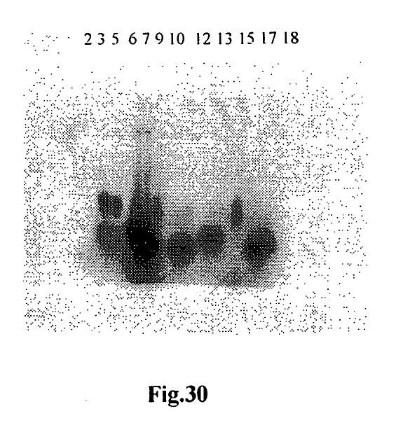

FIG. 32 & 33 Illustrates that use of both oligonucleotide primers of amplification reaction as fluorophore labeled oligonucleotide primers (labeled near 3′ end) does not affect PCR amplification reaction.

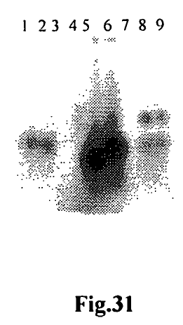

FIG. 32 Ethidium Bromide stained gel picture, Lane 1 & 3 Primer seq nos 19 & 20 0.35 □M each, 100 ng L. donovani DNA, Lane 5 Primer seq nos 10 and 13 each 0.18 □M, 100 ng L. donovani DNA, Lane 7 and 9 Primer sequence nos.20 and 21 0.35 □M each, 100 ng L. donovani DNA.

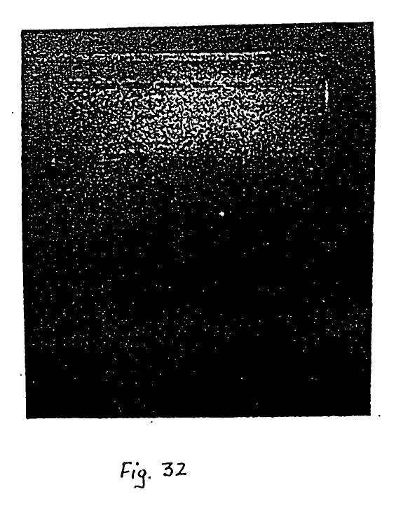

FIG. 33 is same as FIG. 32 but in this case [□³²P] dATP was used for labeling the products.

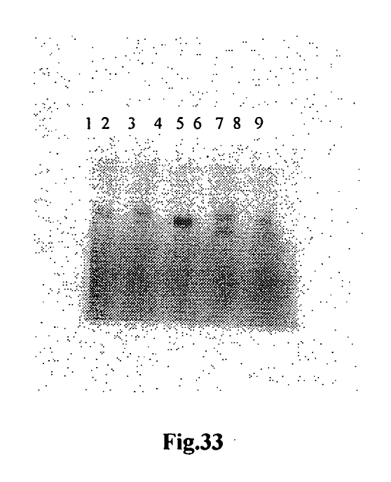

FIG. 34 Detection of an amplification product as by fluorescence resonance energy transfer (FRET) between FAM and JOE on two oligonucleotide primers.

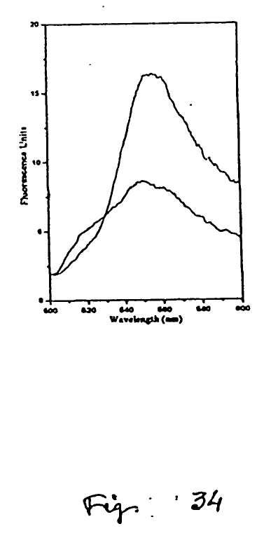

FIG. 35. Illustrates use of hair-pin quenched oligonucleotide reverse primer labeled with an acceptor fluorphore FAM near 3′ end and a quencher DABCYL at 5′ end and a donor fluorophore FAM labeled forward primer resulted in higher FRET signal to noise ratio.

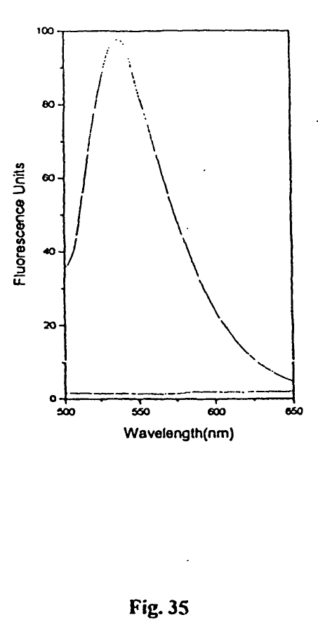

FIG. 36. Illustrates reduction of noise from primer dimer formation in FRET based detection or quantitation of nucleic acid target sequence or amplification product in amplification reaction.

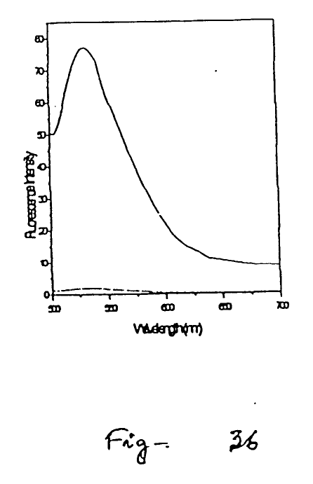

What is claimed:

1-62. (canceled)

63. A simple and improved method of detection and/or quantitation of target nucleic acid sequence comprising (i) providing at least two oligonucleotides as a pair of primers for amplification of said target sequence; (ii) subjecting the target sequence to amplification such that the 3′ ends of said pair of primers are on two opposite strands and separated from one another by 0-25 nucleotide pairs in the final amplification product; and (iii) carrying out denaturation step and at least a selective annealing step in each cycle.

64. The method of claim 63 wherein said denaturation carried out is less than 20 seconds preferably less than 10 seconds, said annealing carried out is less than 15 seconds preferably less than 5 seconds and extension of less than 10 seconds preferably 0 seconds in each cycle for high through put PCR or nucleic acid target analysis.

65. The method of claim 63 wherein one of the two oligonucleotides of the nucleic acid amplification reaction is labeled at or near preferably near 3′ end with a fluorescent or luminescent signaling moiety kept quenched when the labeled oligonucleotide is not incorporated into the amplification product, the same labeled oligonucletide, when incorporated into the amplification product adapted to generate signal through removal of quenching by separating quencher from the signaling moiety.

66. The method of claim 63 wherein a first oligonucleotide is labeled with a donor MET moiety at or near preferably near 3′ end preferably within 2-10 nucleotides away from 3′ end and the second oligonucleotide is labeled with an acceptor MET moiety, at or near preferably near 3′ end within 2-10 nucleotides away from 3′ end, the said donor and acceptor MET moieties belonging to a molecular energy transfer pair and so configured that the donor and the acceptor moieties come within MET distance, 10-80 Angstrom or the nucleotides to which the MET moieties are attached are separated by 2-20 nucleotides preferably by 4-12 nucleotides.

67. The method of claim 66 wherein the labeled oligonucleotides are selected from linear, hair-pin or otherwise configuration.

68. The method of claim 66 wherein the oligonucleotide labeled with the acceptor moiety or both the oligonucleotides labeled separately with donor or acceptor moiety are provided in quenched condition with quencher or quenchers or by providing them with hair-pin stem structure such that the emission energy of the acceptor or both the acceptor and the donor remain quenched when not incorporated into the amplification product.

69. The method of claim 68 wherein said quenching is achieved following anyone of:
a. at least the oligonucleotide labeled with the acceptor provided in a hair-pin quenched configuration, where the acceptor is provided quenched with a quencher or both the donor as well as the acceptor labeled oligonucleotides are provided in hair-pin quenched configuration so that both the donor and the acceptor moieties are provided quenched with two separate quenchers, the quenchers are provided on the same oligonucleotides and attached at or near the respective 5′ ends, the quencher and the acceptor or the donor are on two opposite strands of the stem structure and part of the same oligonucleotide; and in the event of formation of hair-pin stem structure the nucleotide to which the donor or the acceptor moiety is attached is complementary and opposite to the nucleotide to which the quencher is attached or the nucleotide to which the donor or the acceptor moiety is attached and the complement of the nucleotide to which the quencher is attached are within five nucleotides, the donor labeled and or acceptor labeled hair-pin quenched oligonucleotides remain quenched when not incorporated into the amplification product;
b. using additional one or two oligonucleotides as the case may be each being labeled separately at or near 5′ end with suitable quencher for the acceptor or the donor MET moiety such that one member of the quencher labeled additional oligonucleotide is fully or partly complementary to the acceptor labeled oligonucleotide resulting in quenching of the acceptor when the acceptor labeled oligonucleotide is not incorporated into the amplification product and the second member of the quencher labeled additional oligonucleotide is fully or partly complementary to the donor labeled oligonucleotide resulting in quenching of the donor when the donor labeled oligonucleotide is not incorporated into the amplification product ; and
c. by providing the acceptor labeled oligonucleotide linked to another suitable oligonucleotide complementary partly or fully to this acceptor labeled oligonucleotide and labeled with a quencher at or near its 5′ end through a non-nucleotide organic linker or linker and spacer or by providing both the acceptor and donor labeled oligonucleotides linked to two separate additional suitable oligonucleotides fully or partly complementary to the acceptor and donor labeled oligonucleotides respectively through non-nucleotide organic linkers or linkers and spacers and labeled at or near their 5′ ends with two quenchers respectively so that the quenchers can quench the acceptor and the donor when the acceptor and the donor labeled oligonucleotides are not incorporated into the amplification product.

70. The method of claim 63 wherein a first oligonucleotide primer pair selected to amplify a first segment of the target nucleic acid is used at appropriate concentrations, a second oligonucleotide primers selected to amplify a second segment of the first segment at appropriate concentration used in nested PCR when the second oligonucleotide primer pair is any of the labeled oligonucleotide primer pairs of claim 65.

71. The method of claim 63 wherein a first oligonucleotide primer pair selected to amplify a first segment of the target nucleic acid is used at appropriate concentration, where one of the said oligonucleotide primer pair is a first member of the primer pairs of claim 63 which can be either labeled or unlabeled, a third oligonucleotide unlabeled or suitably labeled for MET and designed to amplify a second segment of the first segment in association with the above first member is nested with signal being generated on said selective amplification of the target nucleic acid.

72. The method of claim 63, wherein said nucleic acid amplification reaction comprise any known nucleic acid amplification reactions preferably polymerase chain reaction comprising the steps of adding a polymerase, reaction buffer, deoxy nucleoside triphosphates in addition to the effective amounts of amplification primers to the sample, cycling the sample, between at least a denaturation temperature and an annealing temperature, exciting the reaction mixture with the donor exciting radiation or light, measuring the emission of the acceptor MET moiety, optionally that of the donor.

73. The method of claim 63, wherein said step of amplifying the target sequence comprise a nucleic acid amplification reaction carried out using one labeled oligonucleotide as one of the two amplification primers of the target sequence amplification reaction along with the other unlabeled primer and a third labeled oligonucleotide for the amplification product, the said labeled oligonucleotide primer being labeled at or near 3′ end with a donor or an acceptor MET moiety of a donor-acceptor MET pair and the said third oligonucleotide being labeled at or near 3′ end respectively with an acceptor or donor MET moiety of the above MET pair such that upon successful amplification of the target sequence the labeled primer gets incorporated into one of the two strands of the amplification product and the third labeled oligonucleotide hybridizes to this strand of the amplification product into which the labeled oligonucleotide primer get incorporated thus bringing the donor and the acceptor MET moieties within a MET distance 0-20 nucleotides of one another preferably between 1-10/15 resulting in MET between the two moieties; the above said amplification reaction comprising the steps of adding polymerase, reaction buffer, deoxy nucleoside triphosphates in addition to the effective amounts of the amplification primers to the samples, cycling the sample between at least a denaturation temperature and an elongation temperature, exciting the reaction mixture with the donor exciting radiation or light, measuring the emission of the acceptor MET moiety and optionally the reduction in donor emission, thus allowing detection of nucleic acid target without creating inhibition to amplification reaction and signal measurement without loss of signal.

74. The method of claim 63, wherein
a. at least the oligonucleotide labeled with the acceptor is provided in quenched configuration such that the acceptor remains quenched when the acceptor labeled oligonucleotide is not incorporated into or not hybridized to the amplification product, thus reducing the background and remains unquenched in open configuration of the oligonucleotide producing signal when incorporated into or hybridized to the amplification product;
b. the amplified sample is illuminated with light absorbed by the donor MET moiety; and
c. monitoring the sensitized emission from the acceptor and optionally emission from donor of the MET pair moieties.

75. The method of claim 63, wherein a first oligonucleotide of linear or hair-pin configuration labeled with a donor moiety at or near preferably near its 3′ end and a second oligonucleotide singly labeled at or near its 3′ end with an acceptor moiety capable of absorbing the energy or light emitted by the donor, where the acceptor is selected from a fluorophore or a quencher preferably a quencher including DABCYL or its analogue or nanogold particle black hole quencher, the donor moiety of the first oligonucleotide kept quenched when the first oligonucleotide is not incorporated into the amplification product either by providing a third oligonucleotide fully or partly complementary to the first oligonucleotide seperately or linked to first oligonucleotide through an organic non-nucleotide linker and labeled at or near its 5′ end with a quencher moiety or by providing the first donor labelled oligonucleotide as hair-pin oligonucleotide with a quencher at or near its 5′ ends so configured that the quencher comes in close proximity to the donor moiety in its stem structure, the quencher is selected to be capable of absorbing the energy or light emitted by the donor, and selected to be a fluorophore or a non-radiative quencher preferably a quencher including DABCYL or its analogue, nanogold particle, black hole quencher, and not excluding others, the first and second oligonucleotides are the two primers of nucleic acid amplification reaction and are used such that the emission of the donor is quenched by the quencher/acceptor on the second oligonucleotide only in case of formation of primer dimer but in case of specific amplification product formation the above said quencher/acceptor of second oligonucleotide remains at least 10 bases away from the donor moiety incorporated into the amplification product through the first oligonucleotide and at the same time the quencher of first oligonucleotides remains at least 10 bases away from the donor moiety incorporated into the amplification product through the first oligonucleotides thus allowing the donor moiety to emit its characteristic energy or light and signal generation for the detection or quantitation of a target nucleic acid sequence with increased signal to noise.

76. The method of claim 63, where a first oligonucleotide labeled with a donor-1 moiety at or near preferably near its 3′ end and a second oligonucleotide singly labeled at or near its 3′ end with an acceptor moiety capable of absorbing the energy or light emitted by the donor-1 and a third oligonucleotide singly labeled with a donor-2 moiety at or near its 5′ end are provided, the donor-1 is capable of absorbing the energy or light emitted by the donor-2, the third oligonucleotide is fully or partly complementary as long as the complementarity is maintained such that the donor-2 moiety remains quenched by the donor-1 moiety when the first oligonucleotide is not incorporated into the amplification product; on incorporation of the first oligonucleotide and the second oligonucleotide into the amplification product the donor-2 moiety labeled third oligonucleotide gets separated from the donor-1 labeled first oligonucleotide and the emission of the donor-2 moiety is measured.

77. The method of claim 63, wherein said oligonucleotides are of the length 10 to 40 bases preferably 15-30 bases and the said hair-pin oligonucleotides comprise anyone of the following:
a. a first oligonucleotide 10-40 bases long preferably 15-30 bases long fully complementary to the target nucleic acid sequence at the 5′ end of which is attached a 5-9 bases long second oligonucleotide which may or may not be partially or fully complementary to the target sequence but fully complementary to the 3′ end of the first oligonucleotide thus forming a stem and loop structure;
b. a first oligonucleotide of length between 15-40 preferably 15-30 bases fully complementary to the target nucleic acid sequence at the 5′ end of which is attached a second oligonucleotide of length 2 to 12 bases and again at the 5′ end of the second oligonucleotide is attached a third oligonucleotide of length 5-9 bases, the second and the third oligonucleotide may or may not be partly or fully complementary to the target nucleic acid sequence but the third oligonucleotide being fully complementary to 5-9 bases at or near the 3′ end of the first oligonucleotide thus forming stem and loop structure;
c. a first oligonucleotide of length between 15-40 bases preferably between 15-30 bases fully complementary to the target nucleic acid sequence at the 5′ end of the said first oligonucleotide is attached a second oligonucleotide of length 5-9 bases and at the 3′ end of the said first oligonucleotide is attached a third oligonucleotide of length 5-9 bases, the second and the third oligonucleotides being fully complementary to each other, may or may not be fully or partly complementary to the target nucleic acid sequence thus forming a stem and a loop structure of said hair-pin oligonucleotide;
d. a first oligonucleotide of length between 15-50 preferably 15-30 bases fully complementary to the target nucleic acid sequence at the 5′ end of a first which is attached a second oligonucleotide of length 10 to 30 bases through a non-nucleotide organic linker the second oligonucleotide may or may not be partly or fully complementary to the target nucleic acid sequence but the second oligonucleotide being complementary to the bases at or near the 3′ end of the first oligonucleotide.

78. The method of claim 63, wherein in case of quenched hair-pin, linear or otherwise oligonucleotide the nucleotide to which the donor or acceptor MET moiety is attached is either opposite or within 5 nucleotides away from the nucleotide to which the quencher for the respective donor or acceptor is attached, the donor/acceptor MET moiety and the quencher being on two opposite stands of the stem.

79. The method of claim 63, wherein said oligonucleotides are selected from DNA or RNA or chimeric mixtures or derivatives or modified versions thereof adapted for priming the amplification reaction or hybridizing to the amplified product and are deoxy oligonucleotides, oligonucleotide or peptide nucleic acid or modified oligonucleotides, the target nucleic acid sequence being selected from genomic DNA, mRNA, RNA, cDNA, chemically synthesized DNA or RNA.

80. The method of claim 63, wherein said oligonucleotides are amplification primers (forward and reverse) of polymerase chain reaction (PCR), reverse transcription PCR (RT-PCR), allele specific PCR, methylation status PCR, in situ PCR, Triamplification, Nucleic acid sequence based amplification, immuno PCR and not excluding others.

81. The method of claim 63 used in real time RNA expression profiling by simultaneously quantitating large number of mRNAs or cDNAs using preferably PCR, RT-PCR, NASBA by using suitably labeled oligonucleotide primer pairs selected from individual mRNAs or cDNAs.

82. The method of claim 63 used in high throughput nucleic acid amplification reactions including PCR, RT-PCR and NASBA comprising providing first oligonucleotide amplification primer for each mRNAs or cDNAs in large number from a pool selected from the sequences near the 5′ ends of individual mRNAs or cDNAs and providing as second amplification primer a single common oligonucleotide primer (common for all mRNAs or cDNAs in the pool or sample) complementary to a sequence joined or ligated to 5′ end of all mRNAs or cDNAs synthesized by reverse transcription in the pool or sample prior to the subjection to the amplification reaction the oligonucleotide primers being said labeled oligonucleotides primer pairs either first oligonucleotide amplification primer is dual labeled quenched primer of the invention and the second common amplification primer is an unlabeled oligonucleotide or the first oligonucleotide amplification primer is labeled at or near preferably near 3′ end with a donor or an acceptor MET moiety and the second common oligonucleotide amplification primer is labeled at or near preferably near 3′ end with an acceptor or donor MET moiety respectively which can also be provided quenched.

83. The method of claim 63 used in high throughput nucleic acid amplification reactions including PCR, RT-PCR and NASBA comprising providing first oligonucleotide amplification primer for each mRNAs or cDNAs in large number from a pool selected from the sequences near the 3′ or 5′ ends of a restriction site of cDNAs of individual mRNAs and providing as second amplification primer a single common oligonucleotide primer (common for all mRNAs or cDNAs in the pool or sample) complementary to a sequence joined or ligated to 3′ and 5′ ends of the restriction fragments of all cDNAs synthesized by reverse transcription in the pool or sample prior to the subjection to the amplification reaction the oligonucleotide primers being said labeled oligonucleotides primer pairs, either first specific oligonucleotide amplification primer is dual labeled quenched primer of the invention and the second common amplification primer is an unlabeled oligonucleotide or the first oligonucleotide amplification primer is labeled at or near preferably near 3′ end with a donor or an acceptor MET moiety and the second common oligonucleotide amplification primer is labeled at or near preferably near 3′ end with an acceptor or donor MET moiety respectively, which can also be provided quenched.

84. The method of claim 63 used in RNA splice variant detection, wherein the target nucleotide sequence is a mRNA or a cDNA and the labeled oligonucleotides are either amplification primers (forward and reverse) of many nucleic acid amplification reactions including polymerase chain reaction (PCR), Reverse transcription polymerase chain reaction (RT-PCR), NASBA, one from 3′ end of one exon and the other from 5′ end of the adjacent exon or one of the two amplification primers of many nucleic acid amplification reactions including PCR, RT-PCR from 3′ end of one exon and a probe complementary to 5′ end of the adjacent exon.

85. The method of claim 63 wherein the detectable signal emitted by the acceptor MET moiety/MET moiety is sizeable and more intense than the signal emitted by the same if there is no MET and the oligonucleotides are so designed that MET moieties come in right proximity such that MET between donor and acceptor moieties can occur and FRET is a preferred form of MET.

86. The method of claim 63 wherein the target nucleic acid sequence is an amplification product or the sequence of infectious disease agent, or genomic sequence of a human, animal, plant or any other organism mutation of which is implicated to the presence of a disorder or disease, or a human, animal or plant genomic sequence, the presence or absence of which is implicated to a disorder or disease, or a human, animal or plant genomic sequence, the presence or absence of which is implicated to susceptibility to an infectious agent, or a plant or any living organism genomic sequence the presence or absence of which is implicated to a genetic trait or genotyping of the plants, or the living organism, or a genomic sequence of an infectious agent, the presence or absence of which is implicated to strain typing.

87. The method of claim 63 wherein the donor and acceptor pair moieties are selected from any of the donor-acceptor MET\FRET pairs and the donor moiety is preferably selected from the group consisting of fluorescein, carboxyfluoroscein (FAM), coumarin, 5-(2′ amino ethyl) amino napthlein-1-sulphonic acid (EDANS), rhodamine, anthranilamide, Reactive Red-4,europium and terbium chelate derivatives, a combination of an organic moiety having a large extinction coefficient of absorption and a fluorophore preferably of high quantum yield. and the said acceptor moiety is preferably selected from the group consisting of fluorescein, fluorescein derivatives like JOE and others, ethidium, texas red, eosin nitrotyrosine, malachite green, pyrene butyrate, Cy-3 dye, Cy-5 dye , DABCYL, DABCYL derivatives, rhodamine, rhodamine derivatives, nanogold black hole quencher and the quencher is selected from the group consisting of DABCYL and its derivatives, rhodamine, nanogold particles, black hole quencher and many other acceptor moieties.

88. The method of claim 63, wherein the detection and/or quantitation of amplified target nucleic acid is accomplished by providing double stranded DNA binding fluorescent dye selected from the group preferably consisting of ethidium bromide, CYBER™ GREEN I, pico green, acridine orange, thiazole orange Yo PRO-1 and chromomycin A3.

89. The method of claim 63, wherein the detection and/or quantitation of amplified target nucleic acid sequences is accomplished by providing the first oligonucleotide primer being labeled with a binding moiety preferably selected from biotin, magnetic particle and microsphere and a hapten or attached to an anchor oligonucleotide directly or through a linker which can be respectively captured by streptavidin or magnet or centrifugation or anti-hapten antibody, capture oligonucleotide or the like and the second oligonucleotide primer being labeled with a signaling moiety like a fluorophore, rare earth metal chelate, biotin or a hapten, the hapten being detected utilizing antihapten antibody-enzyme conjugate, steptavidin-enzyme conjugate and enzyme substrate, and other conjugates or by using unlabeled second oligonucleotide primer and providing fluorescently labeled nucleotide in appropriate concentration in the reaction mixture.

90. The method of claim 63, wherein the higher signal to noise ratio improvement is achieved by applying hair-pin quenched labeled oligonucleotides of the invention and selecting an amplification product of the size, the size of the forward amplification primer plus the size of the reverse amplification primer plus 0-25 bases in the detection of a nucleic acid target sequence using MET/FRET between donor & acceptor moieties.

91. The method of claim 63, comprising multiplexing involving multiple pairs of MET labeled oligonucleotides for detection or quantification of multiple targets.

92. The method of claim 63 comprising heterogenous phase detection wherein one of the two amplification primers of many amplification reactions including PCR, RT-PCR, NASBA is fixed covalently through 5′ end or an internal nucleotide to a solid support through a linker and spacer, and the other amplification primer is in aquaeous phase in contact with the solid phase, the said solid support to which the labeled oligonucleotide is attached is non-porous and transparent or translucent and glass or plastics like polystyrene, polyethylene, polypropylene or dextran and the like and preferably glass or glass wafer.

93. The method of claim 63 for high throughput hetrogeneous phase target nucleic acid detection wherein first of the two amplification primers of many target nucleic acids for many amplification reactions including PCR, RT-PCR, NASBA are fixed covalently through 5′ end or an internal nucleotide to a solid support through a linker and spacer, and the second amplification primers are in aquaeous phase in contact with the solid phase, the said solid support to which the labeled oligonucleotide is attached is non-porous and transparent or translucent and glass or plastics like polystyrene, polyethylene, polypropylene or dextran and the like and preferably glass or glass wafer.

94. The method of claim 63 used in high throughput RNA expression profiling by many nucleic acid amplification reactions including PCR, RT-PCR and NASBA not excluding others by providing first oligonucleotide amplification primer for each mRNAs or cDNAs in large number from a pool selected from the sequences near the 5′ ends of individual mRNAs or cDNAs, the said first oligonucleotide primers fixed covalently through 5′ end or an internal nucleotide to a solid support through a linker and spacer, and the second amplification primers are in aqueous phase in contact with the solid phase and providing as second amplification primer a single common oligonucleotide primer (common for all mRNAs or cDNAs in the pool or sample) complementary to a sequence joined or ligated to 5′ end of all mRNAs or cDNAs in the pool or sample prior to the subjection to the amplification reaction the oligonucleotide primers being labeled oligonucleotides primer pairs of the invention, either first oligonucleotide amplification primer is dual labeled quenched primer of the invention and the second common amplification primer is an unlabeled oligonucleotide or the first oligonucleotide amplification primer is labeled at or near preferably near 3′ end with a donor or acceptor MET moiety and the second common oligonucleotide amplification primer is labeled at or near preferably near 3′ end with an acceptor or donor MET moiety respectively and also can be provided quenched.

95. The method of claim 63 used in high throughput RNA expression profiling by many nucleic acid amplification reactions including PCR, RT-PCR and NASBA not excluding others by providing first oligonucleotide amplification primer for each mRNAs or cDNAs in large number from a pool selected from the sequences near the 3′ or 5′ ends of a restriction site of individual mRNAs or cDNAs, the said first oligonucleotide primers fixed covalently through 5′ end or an internal nucleotide to a solid support through a linker and spacer, and the second amplification primers are in aquaeous phase in contact with the solid phase and providing as second amplification primer a single common oligonucleotide primer (common for all mRNAs or cDNAs in the pool or sample) complementary to a sequence joined or ligated to the 3′ and 5′ ends of all mRNAs or cDNAs restriction digested fragments in the pool or sample prior to the subjection to the amplification reaction the oligonucleotide primers being labeled oligonucleotides primer pairs of the invention, either first oligonucleotide amplification primer is dual labeled quenched primer of the invention and the second common amplification primer is an unlabeled oligonucleotide or the first oligonucleotide amplification primer is labeled at or near preferably near 3′ end with a donor or acceptor MET moiety and the second common oligonucleotide amplification primer is labeled at or near preferably near 3′ end with an acceptor or donor MET moiety respectively and also can be provided quenched.

96. The method of claim 63 wherein one or both the primers are provided labeled with a donor or acceptor moiety and a double stranded DNA intercalating dye suitable to act as an acceptor or donor respectively at suitable concentration whereby on successful amplification donor/acceptor labeled primer/primers get incorporated into the amplification product and the double stranded DNA binding (intercalating) dye get intercalated into the amplification product thus bringing it close to the donor or acceptor moiety as the case may be and resulting in MET/FRET which can be measured, more specifically a fluoresecin labeled primer and double stranded DNA binding dye Ethidium bromide are used, where fluorescein is the donor and eithidium act as is the acceptor for FRET to take place between the two.

97. The method of claim 63 used in closed tube format for detection or quantitation of one or more nucleic acid target sequences

98. The method of claim 63 wherein the oligonucleotides used are selected from:
a.5′-GGG GTA CTA CAG CGC CCT GA-3′
```
b. 5′-GGG GTA CTA CAG CGC CCT GA -3′
                                                        |
                                                        FAM
c. 5′-GTC CTG GAA GAT GGC CAT GG -3′
d. 5′-GTC CTG GAA GAT GGC CAT GG -3′
                                                        |
                                                      JOE
e. 5′-ATG GCC ATC GTC CTG GAA GAT GGC CAT GG -3′
f. 5′-DABCYL-ATG GCC ATC GTC CTG GAA GAT GGC CAT GG -3′
                                                                                              |
                                                                                            JOE
g. 5′-DABCYL-ATG GCC ATC GTC CTG GAA GAT GGC CAT GG -3′
                                                                                              |
                                                                                            FAM
h. 5′-GCT CAT GGC GCC TGC CTG G -3′
                                                      |
                                                    DABCYL
i. 5′-ATG GCC ATG TCC TGG AAG ATG GCC ATG G -3′
j. 5′-GGG GTA CTA CAG CGC CCT -3′
                                                      |
                                                      FAM
k. 5′-GTC CTG GAA GAT GGC CAT GG -3′
                                                        |
                                                    Rhod
l. 5′-GTC CTG GAA GAT GGC CAT GG -3′
                                    |
                                  JOE
m. 5′GGC AAT GAA AAG CCA CTT CT -3′
as a forward primer to amplify a 50 base pair segment (base position
23,565-23,614) of E. coli genome
n. 5′TTA ACC GGC GAT TGA GTA CC -3′
as a reverse primer to amplify a 50 base pair segment (base position
23,565-23,614) of E. coli genome.
```

99. A kit for use in method of analogous detection and/or quantitation of target nucleic acid sequence or sequences present in a sample comprising
a. a polymerase or polymerases
b. at least two oligonucleotides as a pair of primers for amplification of said target sequence such that after amplifiction the 3′ ends of said pair of primers are on two opposite strands and separated from one another by 0-25 nucleotide pairs in the final amplification product;
c. deoxynucleotides in solution (water or buffer) or lyophilized;
d. a reaction buffer for the nucleic acid amplification reaction.

100. The kit of claim 99 wherein the oligonucleotides as pair of primers are suitably labeled separately at or near preferably near their 3′ ends with a donor or an acceptor MET/FRET moiety and contains free 3′ hydroxy group for polymerase to extend, the said donor and acceptor MET moieties belonging to a molecular energy transfer pair and so configured that the donor and the acceptor moieties come close within MET/FRET distance in the amplification product and the nucleotides to which the donor and acceptor moieties are attached are 0-25 nucleotide pairs away.

101. The kit of claim 100 wherein at least the acceptor moiety of the acceptor MET moiety labeled oligonucleotide primer is provided quenched when the same is not incorporated into the amplification product or both the donor and acceptor MET moieties of the respectively labeled oligonucleotide primers are provided quenched when the same are not incorporated into the amplification product, the above quenched oligonucleotide primers being the oligonucleotide primers of claim 69.

102. The kit of claim 99 wherein additionally positive control template and suitable MET/FRET labeled primers are also included as control for amplification reaction.

103. The kit of claim 99 wherein the first oligonucleotide is labeled near 3′ end with a donor MET/FRET moiety and a double stranded DNA intercalating dye capable of absorbing energy or light emitted by the donor moiety and emitting energy or light is also provided.

104. The kit of claim 99 wherein the first oligonucleotide is labeled near 3′ end with an acceptor MET/FRET moiety and a double stranded DNA intercalating dye capable of emitting energy or light on illumination is provided such that the acceptor moiety is capable of absorbing the energy or light emitted by the intercalating dye and emitting energy or light.

105. The kit of claim 99 comprising multiple oligonucleotide sets of claim for detection and/or/quantitation of multiple target sequences.

106. A kit for the detection of target nucleic acid sequences or sequences using the oligonucleotides used in the methods of claim 67 wherein the detection and/or quantitation of amplified target nucleic acid sequences is accomplished by providing the first oligonucleotide primer being labeled at or near 5′ end with a binding moiety preferably biotin, or magnetic particle or microsphere or a hapten or the like or attached to an anchor oligonucleotide directly or through a linker which can be respectively captured by streptavidin or magnet or centrifugation or anti-hapten antibody, capture oligonucleotide or the like and the second oligonucleotide primer being labeled with a signaling moiety like a fluorophore, rare earth metal chelate, biotin or a hapten, the hapten being detected utilizing. antihapten antibody-enzyme conjugate, steptavidin-enzyme conjugate and enzyme substrate, and other conjugates or by using unlabeled second oligonucleotide primer and providing fluorescently labeled nucleotide in the reaction mixture in appropriate concentration.

107. A kit or kits for the detection of target nucleic acid sequences providing all or more components using the oligonucleotides for detection used in methods of claim 67 wherein the detection and/or quantitation of amplified target nucleic acid is accomplished by providing double stranded DNA binding fluorescent dye selected from the group consisting of eithidium bromide, CYBER TM GREEN I, pico green, acridine orange, thiazole orange Yo PRO-1 and chromomycin A3 but not excluding others.

108. The kit of claim 99 wherein the oligonucleotides used are selected from:
```
a. 5′-GGG GTA CTA CAG CGC CCT GA -3′
b. 5′-GGG GTA CTA CAG CGC CCT GA -3′
                                                        |
                                                        FAM
c. 5′-GTC CTG GAA GAT GGC CAT GG -3′
d. 5′-GTC CTG GAA GAT GGC CAT GG -3′
                                                        |
                                                        JOE
e. 5′-ATG GCC ATC GTC CTG GAA GAT GGC CAT GG -3′
f. 5′-DABCYL-ATG GCC ATC GTC CTG GAA GAT GGC CAT GG -3′
                                                                                              |
                                                                                            JOE
g. 5′-DABCYL-ATG GCC ATC GTC CTG GAA GAT GGC CAT GG -3′
                                                                                              |
                                                                                            FAM
h. 5′-GCT CAT GGC GCC TGC CTG G -3′
                                                      |
                                                  DABCYL
i. 5′-ATG GCC ATG TCC TGG AAG ATG GCC ATG G -3′
j. 5′-GGG GTA CTA CAG CGC CCT -3′
                                                        |
                                                      FAM
k. 5′-GTC CTG GAA GAT GGC CAT GG -3′
                                                        |
                                                      Rhod
l. 5′-GTC CTG GAA GAT GGC CAT GG -3′
                                    |
                                  JOE
m. 5′GGC AAT GAA AAG CCA CTT CT -3′
as a forward primer to amplify a 50 base pair segment (base position
23,565-23,614) of E. coli genome
n. 5′TTA ACC GGC GAT TGA GTA CC -3′
as a reverse primer to amplify a 50 base pair segment (base position
23,565-23,614) of E. coli genome.
```

109. A method of manufacture of a kit for use in method of analogous detection and/or quantitation of target nucleic acid sequence or sequences present in a sample comprising
a. providing a polymerase or polymerases
b. providing a first oligonucleotide of sequence complementary to the nucleotide sequence flanking a target nucleotide sequence suitably labeled with a donor MET/FRET moiety at or near 3′ end.
c. providing a second oligonucleotide of sequence at 5′ end of the first nucleotide sequence complementary to the nucleotide sequence flanking the target nucleotide sequence or the segment of the target nucleotide sequence suitably labeled with an acceptor MET/FRET moiety at or near 3′ end.
d. providing deoxy nucleotides in solution (water or buffer) or lyophilized.
e. providing a reaction buffer for the nucleic acid amplification reaction.
wherein the first and the second oligonucleotide sequences comprise the two primers (forward and reverse) of many nucleic acid amplification reactions and adapted to generate a detectable signal if the two oligonucleotides get incorporated into two opposite strands of amplified product and come in right proximity an the first and second oligonucleotides are any of the quenched oligonucleotide primers of claim 63.

110. A method for high throughput RNA expression preferably for analysis of absolute quantating of mRNAs carried out in homogenous or heterogeneous phase using the method of detection/quantification of target nucleic acid sequence as claimed in claim 63.

111. The method of claim 63 for the heterogeneous mutation detection comprising two amplification primer oligonucleotides of the invention one being labeled with a donor MET moity near 3′ end and the other being labeled with an acceptor MET moiety near 3′ end, carrying out target amplification reaction and thermal denaturation analysis of the amplification product or products thus amplified and the same method where the labeled oligonucleotides are also provided in quenched configuration.

112. The method of claim 63 used in high throughout nucleic acid amplification reactions including PCR, RT-PCR and not excluding others.

113. The method of claim 63 wherein the donor moiety is a combination of an organic moiety having a large extinction coefficient of absorption and a fluorophore preferably of high quantum yield and extinction coefficient.

114. The method of claim 63 wherein the higher signal to noise ratio is achieved by applying hair-pin quenched labeled oligonucleotides of the invention in ligase chain reaction.

115. The method of claim 63 wherein the higher signal to noise ratio improvement is achieved by applying hair-pin quenched or otherwise quenched labeled oligonucleotides of the invention in the detection of a nucleic acid target sequence using MET/FRET between donor and acceptor moieties on two oligonucleotides designed against one strand of the target sequence.

116. The method of claim 63 wherein one or both the amplification primers are labeled at or near 3′ end with acceptor or donor moiety or moieties and one of the four deoxynucleotides is provided labeled with the donor or acceptor moiety respectively in appropriate concentrations and composition, and wherein on incorporation of the acceptor labeled primer or primers and the donor labeled nucleotide into the amplification product there is MET/FRET between the donor and the acceptor.

117. The kit of claim 99 wherein the polymerases are a reverse transcriptase, T7 RNA polymerase and a DNA polymerase.

118. A method of detection of target nucleic acid sequence, a kit used for the same and its process of manufacture substantially as herein described and illustrated with reference to examples and figures and many modifications thereof.


SEQUENCE LISTING (31 sequences)

SEQ ID NO: 1 - DNA, 20 bp, Artificial
  Forward PCR primer for amplification of a target sequence chosen arbitrarily and made from Sequence ID Nos. 3 and 4.
acttaagttagagcgtttgc

SEQ ID NO: 2 - DNA, 20 bp, Artificial
  Forward PCR primer for amplification of a target sequence chosen arbitrarily and made from Sequence ID Nos. 3 and 4.
tggtagtatgtgatttagtc

SEQ ID NO: 3 - DNA, 40 bp, Artificial
  Arbitrarily chosen sequences. Bases 27 to 40 are complementary to basis 31 to 44 of Sequence ID No. 4. DNA polymerase extension of annealed Sequence ID Nos. 3 and 4 results in the target sequence.
tacacttaagttagagcgtttgcgcccactacgacggttg

SEQ ID NO: 4 - DNA, 44 bp, Artificial
  Arbitrarily chosen sequences. Bases 27 to 40 are complementary to bases 31 to 44 of Sequence ID No. 4. DNA polymerase extension of annealed Sequence ID Nos. 3 and 4 results in the target sequence.
gtttttgtggtagtatgtgatttagtcattcaaccgtcgtagtg

SEQ ID NO: 5 - DNA, 20 bp, Artificial
  Forward PCR primer for amplification of a target sequence chosen arbitrarily and made from Sequence ID Nos. 3 and 4. Base t at base position 18 from 5′ end has fluorophore FAM.
acttaagttagagcgtttgc

SEQ ID NO: 6 - DNA, 19 bp, Leishmania donovani
acggagcggctgaaggtgc

SEQ ID NO: 7 - DNA, 27 bp, Leishmania donovani
aggtgcatccacttgtcctgcacctgc

SEQ ID NO: 8 - DNA, 21 bp, Leishmania donovani
aggcagatggcgcctgcctcg

SEQ ID NO: 9 - DNA, 25 bp, Leishmania donovani
atgcggcgctgtagtaccccgcatc

SEQ ID NO: 10 - DNA, 20 bp, Leishmania donovani
ggggtactacagcgccctga

SEQ ID NO: 11 - DNA, 28 bp, Leishmania donovani
atggccatgtcctggaagatggccatgg

SEQ ID NO: 12 - DNA, 29 bp, Leishmania donovani
atggccatcgtcctggaagatggccatgg

SEQ ID NO: 13 - DNA, 20 bp, Leishmania donovani
gtcctggaagatggccatgg

SEQ ID NO: 14 - DNA, 20 bp, Leishmania donovani
ctgcacacggagcggctgaa

SEQ ID NO: 15 - DNA, 20 bp, Leishmania donovani
ggacgagctcatggcgcctg

SEQ ID NO: 16 - DNA, 20 bp, Leishmania donovani
gtcctgttcaccttccactg

SEQ ID NO: 17 - DNA, 19 bp, Leishmania donovani
gctcatggcgcctgcctcg

SEQ ID NO: 18 - DNA, 19 bp, Leishmania donovani
gcgtgtagtaccccgcatc

SEQ ID NO: 19 - DNA, 20 bp, Leishmania donovani
ggggtactacagcgccctga

SEQ ID NO: 20 - DNA, 20 bp, Leishmania donovani
gtcctggaagatggccatgg

SEQ ID NO: 21 - DNA, 18 bp, Leishmania donovani
ggggtactacagcgccct

SEQ ID NO: 22 - DNA, 29 bp, Leishmania donovani
atggccatcgtcctggaagatggccatgg

SEQ ID NO: 23 - DNA, 29 bp, Leishmania donovani
atggccatcgtcctggaagatggccatgg

SEQ ID NO: 24 - DNA, 19 bp, Leishmania donovani
gctcatggcgcctgcctcg

SEQ ID NO: 25 - DNA, 20 bp, Leishmania donovani
gtcctggaagatggccatgg

SEQ ID NO: 26 - DNA, 20 bp, Leishmania donovani
gtcctggaagatggccatgg

SEQ ID NO: 27 - DNA, 20 bp, Escherichia coli
tgaattcaatctcgcaaacg

SEQ ID NO: 28 - DNA, 26 bp, Escherichia coli
atcggatcccaaatgcctgaggccag

SEQ ID NO: 29 - DNA, 20 bp, Escherichia coli
ggcaatgaaaagccacttct

SEQ ID NO: 30 - DNA, 20 bp, Escherichia coli
ttaaccggcgattgagtacc

SEQ ID NO: 31 - DNA, 20 bp, Escherichia coli
agccttatgacgtgcagctt